(19) United States
(12) Patent Application Publication
(10) Pub. No.: US 2002/0092041 A1
(43) Pub. Date: Jul. 11, 2002

(54) PROCEDURES AND MATERIALS FOR CONFERRING DISEASE RESISTANCE IN PLANTS

(76) Inventors: PAMELA C. RONALD, DAVIS, CA (US); GUO-LIANG WANG, DAVIS, CA (US); WEN-YUANG SONG, DAVIS, CA (US); VERONIQUE SZABO, DAVIS, CA (US); SCOT HULBERT, MANHATTAN, KS (US); TODD RICHTER, KOKOMO, IN (US)

Correspondence Address: PIONEER HI-BRED INTERNATIONAL INC., 7100 N.W. 62ND AVENUE, P.O. BOX 1000, JOHNSTON, IA, 50131, US

(21) Appl. No.: 08/910,386

(22) Filed: Aug. 13, 1997

(51) Int. Cl. A01H 5/00; C12N 15/82

(52) U.S. Cl. 800/301; 800/279

(57) ABSTRACT

The present invention provide nucleic acids encoding polypeptides which confer resistance to Xanthomonas spp and other pathogens. The nucleic acids can be used to produce transgenic plants resistant to the pathogen.

This application is related to U.S. patent application No. 08/587,680, filed Jan. 17, 1996, which is a continuation in part of copending U.S. patent application No. 08/567,375, filed Dec. 4, 1995, which is a continuation in part of U.S. provisional patent application No., 60/004,645. The '680 application is also a continuation in part of copending U.S. patent application No. 08/475,891, filed Jun. 7, 1995, which is a continuation in part of copending U.S. patent application No. 08/373,374, filed Jan. 17, 1995. These applications are incorporated herein by reference.

### STATEMENT AS TO RIGHTS TO INVENTIONS MADE UNDER FEDERALLY SPONSORED RESEARCH AND DEVELOPMENT

This invention was made with Government support under Grant No. GM47907, awarded by the National Institutes of Health and Grant No. 9300834, awarded by the United States Department of Agriculture. The Government has certain rights in this invention.

### FIELD OF THE INVENTION

The present invention relates generally to plant molecular biology. In particular, it relates to nucleic acids and methods for conferring disease resistance in plants.

### BACKGROUND OF THE INVENTION

Loci conferring disease resistance have been identified in many plant species. Genetic analysis of many plant-pathogen interactions has demonstrated that plants contain loci that confer resistance against specific races of a pathogen containing a complementary avirulence gene. Molecular characterization of these genes should provide means for conferring disease resistance to a wide variety of crop plants.

Those plant resistance genes that have been characterized at the molecular level fall into four classes. One gene, Hm1 in corn, encodes a reductase and is effective against the fungal pathogen Cochliobolus carbonum (Johal et al. Science 258:985-987 (1992)). In tomato, the Pto gene confers resistance against Pseudomonas syringae that express the avrPto avirulence gene (Martin et al. Science 262:1432 (1993)). The predicted Pto gene encodes a serine threonine protein kinase. The tomato Cf-9 gene confers resistance to races of the fungus Cladosporium fulvum that carry the avirulence gene Avr9 (Jones et al. Science 266:789-793 (1994). The tomato Cf-9 gene encodes a putatitive extracellular LRR protein. Finally, the RPS2 gene of Arabidopsis thaliana confers resistance to P. syringae that express the avrRpt2 avirulence gene (Bent et al. Science 265:1856-1860 (1994)). RPs2 encodes a protein with an LRR motif and a P-loop motif.

Bacterial blight disease caused by Xanthomonas spp. infects virtually all crop plants and leads to extensive crop losses worldwide. Bacterial blight disease of rice (Oryza sativa), caused by Xanthomonas oryzae pv. oryzae (Xoo), is an important disease of this crop. Races of Xoo that induce resistant or susceptible reactions on rice cultivars with distinct resistance (Xa) genes have been identified. One source of resistance (Xa21) had been identified in the wild species Oryza longistaminata (Khush et al. in Proceedings of the International Workshop on Bacterial Blight of Rice. (International Rice Research Institute, 1989) and Ikeda et al. Jpn J. Breed 40 (Suppl. 1):280-281 (1990)). Xa21 is a dominant resistance locus that confers resistance to all known isolates of Xoo and is the only characterized Xa gene that carries resistance to Xoo race 6. Genetic and physical analysis of the Xa21 locus has identified a number of tightly linked markers on chromosome 11 (Ronald et al. Mol. Gen. Genet. 236:113-120 (1992)). The molecular mechanisms by which the Xa21 locus confers resistance to this pathogen were not identified, however.

Considerable effort has been directed toward cloning plant genes conferring resistance to a variety of bacterial, fungal and viral diseases. Only one pest resistance gene has been cloned in monocots. Since monocot crops feed most humans and animals in the world, the identification of disease resistance genes in these plants is particularly important. The present invention addresses these and other needs.

### SUMMARY OF THE INVENTION

The present invention provides isolated nucleic acid constructs comprising an RRK polynucleotide sequence. The sequences can be rice sequences which hybridize to SEQ ID NOs: 1, 4, 6, 8, 10, or 11 under stringent conditions. Also claimed are sequences from cassava which hybdridize to SEQ ID NO: 13), maize sequences which hybridize to SEQ ID NOs: 15, 16), and tomato (e.g., SEQ ID NOs:17, 19, or 21). Exemplary RRK polynucleotide sequences are Xa21 sequences which encode an Xa21 polypeptide as shown below. The RRK polynucleotides encode a protein having a leucine rich repeat motif and/or a cytoplasmic protein kinase domain. The nucleic acid constructs of the invention may further comprise a promoter operably linked to the RRK polynucleotide sequence. The promoter may be a tissue-specific promoter or a constitutive promoter.

The invention also provides nucleic acid constructs comprising a promoter sequence from an RRK gene linked to a heterologous polynucleotide sequence. Exemplary heterologous polynucleotide sequences include structural genes which confer pathogen resistance on plants.

The invention further provides transgenic plants comprising a recombinant expression cassette comprising a promoter from an RRK gene operably linked to a polynucleotide sequence as well as transgenic plants comprising a recombinant expression cassette comprising a plant promoter operably linked to an RRK polynucleotide sequence. Although any plant can be used in the invention, rice and tomato plants may be conveniently used.

The invention further provides methods of enhancing resistance to Xanthomonas and other pathogens in a plant. The methods comprise introducing into the plant a recombinant expression cassette comprising a plant promoter operably linked to an RRK polynucleotide sequence. The methods may be conveniently carried out with rice or tomato plants.

### Definitions

The term “plant” includes whole plants, plant organs (e.g., leaves, stems, roots, etc.), seeds and plant cells and progeny of same. The class of plants which can be used in the methods of the invention is generally as broad as the class of higher plants amenable to transformation techniques, including both monocotyledonous and dicotyledonous plants.

A “heterologous sequence” is one that originates from a foreign species, or, if from the same species, is substantially modified from its original form. For example, a promoter operably linked to a heterologous structural gene is from a species different from that from which the structural gene was derived, or, if from the same species, one or both are substantially modified from their original form.

An “RRK gene” is member of a new class of disease resistance genes which encode RRK polypeptides which typically comprise an extracellular LRR domain, a transmembrane domain, and a cytoplasmic protein kinase domain (as shown in e.g., Pto and Fen (Martin et al. Plant Cell 6:1543-1552 (1994)). As used herein, an LRR domain is a region of a repeated unit of about 24 residues as described in U.S. Ser. No. 08/587,680, and found in Cf-9). Using the sequences disclosed here and standard nucleic acid hybridization and/or amplification techniques, one of skill can identify members of this class of genes. For instance, a nucleic acid probe from an Xa21 gene detected polymorphisms that segregated with the blast (Pyricularia oryzae) resistance gene (Pi7) in 58 recombinant inbred lines of rice. The same probe also detected polymorphism in nearly isogenic lines carrying xa5 and Xa10 resistance genes.

In some preferred embodiments, members of this class of disease resistance genes can be identified by their ability to be amplified by degenerate PCR primers which correspond to the LRR and kinase domains. For instance, primers have been used to isolate homologous genes in tomato, maize and cassava. The maize gene disclosed here has been genetically mapped to a region associated with resistance to Helminthosporium turcicum. Exemplary primers for this purpose are tcaagcaacaatttgtcaggnca (a/g) at (a/c/t) cc (for the LRR domain sequence GQIP) and taacagcacattgcttgatttnan (g/a) tcncg (g/a) tg (the kinase domain sequence HCDIK). These or equivalent primers are then used to amplify the appropriate nucleic acid using the PCR conditions described below.

An “Xa21 polynucleotide sequence” is a subsequence or full length polynucleotide sequence of an Xa21 gene, such as the rice Xa21 gene, which, when present in a transgenic plant confers resistance to Xanthomonas spp. (e.g., X. oryzae) on the plant. Exemplary polynucleotides of the invention include the coding region of the sequences provided below. An Xa21 polynucleotide is typically at least about 3100 nucleotides to about 6500 nucleotides in length, usually from about 4000 to about 4500 nucleotides.

An “Xa21 polypeptide” is a gene product of an Xa21 polynucleotide sequence, which has the activity of Xa21, i.e., the ability to confer resistance to Xanthomonas spp. Xa21 polypeptides, like other RRK polypeptides, are characterized by the presence of an extracellular domain comprising a region of leucine rich repeats (LRR) and/or a cytoplasmic protein kinase domain. Exemplary Xa21 polypeptides of the invention include those described below.

In the expression of transgenes one of skill will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional RRK polypeptide, one of skill will recognize that because of codon degeneracy, a number of polynucleotide sequences will encode the same polypeptide. These variants are specifically covered by the term “RRK polynucleotide sequence”. In addition, the term specifically includes those full length sequences substantially identical (determined as described below) with an RRK gene sequence and that encode proteins that retain the function of the RRK protein. Thus, in the case of rice RRK genes disclosed here, the above term includes variant polynucleotide sequences which have substantial identity with the sequences disclosed here and which encode proteins capable of conferring resistance to Xanthomonas or other plant diseases and pests on a transgenic plant comprising the sequence.

Two polynucleotides or polypeptides are said to be “identical” if the sequence of nucleotides or amino acid residues, respectively, in the two sequences is the same when aligned for maximum correspondence as described below. The term “complementary to” is used herein to mean that the complementary sequence is identical to all or a portion of a reference polynucleotide sequence.

Sequence comparisons between two (or more) polynucleotides or polypeptides are typically performed by comparing sequences of the two sequences over a segment or “comparison window” to identify and compare local regions of sequence similarity. The segment used for purposes of comparison may be at least about 20 contiguous positions, usually about 50 to about 200, more usually about 100 to about 150 in which a sequence may be compared to a reference sequence of the same number of contiguous positions after the two sequences are optimally aligned.

Optimal alignment of sequences for comparison may be conducted by the local homology algorithm of Smith and Waterman Adv. Appl. Math. 2: 482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, FASTA, and TFASTA in the Wisconsin Genetics Software Package, Genetics Computer Group (GCG), 575 Science Dr., Madison, Wis.), or by inspection. “Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 60% sequence identity, preferably at least 80%, more preferably at least 90% and most preferably at least 95%, compared to a reference sequence using the programs described above (preferably BESTFIT) using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%, preferably at least 60%, more preferably at least 90%, and most preferably at least 95%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Another indication that nucleotide sequences are substantially identical is if two molecules hybridize to each other under appropriate conditions. Appropriate conditions can be high or low stringency and will be different in different circumstances. Generally, stringent conditions are selected to be about 5° C. to about 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent wash conditions are those in which the salt concentration is about 0.02 molar at pH 7 and the temperature is at least about 60° C. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. For Southern hybridizations, high stringency wash conditions will include at least one wash in 0.1X SSC at 65° C.

Nucleic acids of the invention can be identified from a cDNA or genomic library prepared according to standard procedures and the nucleic acids disclosed here (typically at least 100 nucleotides to about full length) used as a probe. Low stringency hybridization conditions will typically include at least one wash using 2X SSC at 65° C. The washes are preferrably followed by a subsequent wash using 1X SSC at 65° C.

As used herein, a homolog of a particular RRK gene (e.g., the rice Xa21 genes disclosed here) is a second gene (either in the same species or in a different species) which encodes a protein having an amino acid sequence having at least 25% identity or 45% similiarity to (determined as described above) to a polypeptide sequence in the first gene product. It is believed that, in general, homologs share a common evolutionary past.

### BRIEF DESCRIPTION OF THE DRAWINGS

FIG. 1 shows the genome organization of the seven Xa21 family members and location of 14 transposon-like elements. Cosmid and BAC clones carrying the family members are designated. Wide bars represent predicted coding regions, fine bars represent noncoding regions, introns are indicated by angled lines, and the non-sequenced regions are shown by straight lines. A gap in the sequence of BAC9 is indicated by “//”. Letters refer to names of Xa21 gene family members and arrows indicate direction of ORFs. The 14 transposon-like elements are numbered and represented by closed triangles.

FIG. 2A shows the HC region of the sequenced Xa21 gene family members. Wide bars represent predicted coding regions, and fine bars represent non-coding regions. Start and stop codons are indicated. The 5′ flanking regions and downstream regions are grouped into four and two groups, respectively, and are shown in different colors based on sequence identity. The percentage of DNA sequence identity between promoter regions and between classes is shown to the left and right, respectively. The HC region is indicated by a black bar.

FIG. 2B is a schematic diagram showing a comparison of the predicted amino acid sequences of XA21 and A1. Domains are numbered as follows: I, Presumed signal peptide; II, presumed N terminus; III, LRR; VI, charged; V, presumed transmembrane; VI charged; VII juxtamembrane; VIII, serine/threonine kinase; IX, carboxy tail. The numbers below each domain indicate amino acid identity between XA21 and A1.

FIG. 3A shows family member D and insertion position of Retrofit. Retrofit carries long terminal repeats (LTRs) (small arrows) and a single, large ORF, encoding a protein with the following domains: gag, protease (PR), integrase (IN), reverse transcriptase (RT), and RNase H (RH). The large arrow indicates direction of the ORF.

FIG. 3B shows family member E and insertion position of Truncator. Arrows mark the orientation of the inverted repeats. The deduced amino acid sequences of the tomato resistance genes Cf9 and Pto are shown below. In both FIGS. 3A and 3B, the insertion elements are designated by a hatched bar. The presumed deduced amino acid sequences of members D and E are shown by shaded rectangles. Domains representations are as described in the legend to FIG. 2.

FIG. 4 shows intergenic recombination break point in the Xa21 family members. Boxes represent the ORFs of the designated family members, while narrow boxes represent flanking regions. Same colors indicate a high level of sequence homology. The nucleotides of the presumed recombination break points are indicated in large and bold type. Sequences surrounding the recombination break point are also shown.

### DESCRIPTION OF THE PREFERRED EMBODIMENTS

This invention relates to plant RRK genes, such as the Xa21 genes of rice. Nucleic acid sequences from RRK genes, in particular Xa21 genes, can be used to confer resistance to Xanthomonas and other pathogens in plants. The invention has use in conferring resistance in all higher plants susceptible to pathogen infection. The invention thus has use over a broad range of types of plants, including species from the genera Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Zea, Avena, Hordeum, Secale, Triticum, and, Sorghum.

The Example section below, which describes the isolation and characterization of RRK genes in rice, casava, maize and tomato. The methods used to isolate these genes are exemplary of a general approach for isolating Xa21 genes and other RRK genes. The isolated genes can then be used to construct recombinant vectors for transferring RRK gene expression to transgenic plants.

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989).

The isolation of Xa21 and related RRK genes may be accomplished by a number of techniques. For instance, oligonucleotide probes based on the sequences disclosed here can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ, such as leaf and a cDNA library which contains the RRK gene transcript is prepared from the mRNA. Alternatively, cDNA may be prepared from mRNA extracted from other tissues in which RRK genes or homologs are expressed.

The cDNA or genomic library can then be screened using a probe (typically a degenerate probe) based upon the sequence of a cloned RRK gene such as rice Xa21 genes disclosed here. Probes may be used to hybridize with genomic DNA or cDNA sequences to isolate homologous genes in the same or different plant species.

Alternatively, the nucleic acids of interest can be amplified from nucleic acid samples using amplification techniques. For instance, polymerase chain reaction (PCR) technology to amplify the sequences of the RRK and related genes directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying RRK sequences from plant tissues are generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990), incorporated herein by reference.

Polynucleotides may also be synthesized by well-known techniques as described in the technical literature. See, e.g., Carruthers et al., Cold Spring Harbor Symp. Quant. Biol. 47:411-418 (1982), and Adams et al., J. Am. Chem. Soc. 105:661 (1983). Double stranded DNA fragments may then be obtained either by synthesizing the complementary strand and annealing the strands together under appropriate conditions, or by adding the complementary strand using DNA polymerase with an appropriate primer sequence.

Isolated sequences prepared as described herein can then be used to provide RRK gene expression and therefore Xanthomonas resistance in desired plants. One of skill will recognize that the nucleic acid encoding a functional RRK protein need not have a sequence identical to the exemplified gene disclosed here. In addition, the polypeptides encoded by the RRK genes, like other proteins, have different domains which perform different functions. Thus, the RRK gene sequences need not be full length, so long as the desired functional domain of the protein is expressed. As explained in detail below, the proteins of the invention comprise an extracellular leucine rich repeat domain, as well as an intracellular kinase domain. Modified protein chains can also be readily designed utilizing various recombinant DNA techniques well known to those skilled in the art. For example, the chains can vary from the naturally occurring sequence at the primary structure level by amino acid substitutions, additions, deletions, and the like. Modification can also include swapping domains from the proteins of the invention with related domains from other pest resistance genes. For example, the extra cellular domain (including the leucine rich repeat region) of the proteins of the invention can be replaced by that of the tomato Cf-9 gene and thus provide resistance to fungal pathogens of rice. These modifications can be used in a number of combinations to produce the final modified protein chain.

To use isolated RRK sequences in the above techniques, recombinant DNA vectors suitable for transformation of plant cells are prepared. Techniques for transforming a wide variety of higher plant species are well known and described in the technical and scientific literature. See, for example, Weising et al. Ann. Rev. Genet. 22:421-477 (1988).

A DNA sequence coding for the desired RRK polypeptide, for example a cDNA or a genomic sequence encoding a full length protein, will be used to construct a recombinant expression cassette which can be introduced into the desired plant. An expression cassette will typically comprise the RRK polynucleotide operably linked to transcriptional and translational initiation regulatory sequences which will direct the transcription of the sequence from the RRK gene in the intended tissues of the transformed plant.

For example, a plant promoter fragment may be employed which will direct expression of the RRK in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the RRK gene in a specific tissue or may be otherwise under more precise environmental or developmental control. Such promoters are referred to here as “inducible” promoters. Examples of environmental conditions that may effect transcription by inducible promoters include pathogen attack, anaerobic conditions, or the presence of light.

Examples of promoters under developmental control include promoters that initiate transcription only in certain tissues, such as leaves, roots, fruit, seeds, or flowers. The operation of a promoter may also vary depending on its location in the genome. Thus, an inducible promoter may become fully or partially constitutive in certain locations.

The endogenous promoters from the RRK genes of the invention can be used to direct expression of the genes. These promoters can also be used to direct expression of heterologous structural genes. Thus, the promoters can be used in recombinant expression cassettes to drive expression of genes conferring resistance to any number of pathogens, including fungi, bacteria, and the like.

To identify the promoters, the 5′ portions of the clones described here are analyzed for sequences characteristic of promoter sequences. For instance, promoter sequence elements include the TATA box consensus sequence (TATAAT), which is usually 20 to 30 base pairs upstream of the transcription start site. In plants, further upstream from the TATA box, at positions −80 to −100, there is typically a promoter element with a series of adenines surrounding the trinucleotide G (or T) N G. J. Messing et al., in Genetic Engineering in Plants, pp. 221-227 (Kosage, Meredith and Hollaender, eds. 1983).

If proper polypeptide expression is desired, a polyadenylation region at the 3′-end of the RRK coding region should be included. The polyadenylation region can be derived from the natural gene, from a variety of other plant genes, or from T-DNA.

The vector comprising the sequences from an RRK gene will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

Such DNA constructs may be introduced into the genome of the desired plant host by a variety of conventional techniques. For example, the DNA construct may be introduced directly into the genomic DNA of the plant cell using techniques such as electroporation, PEG poration, particle bombardment and microinjection of plant cell protoplasts or embryogenic callus, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Transformation techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987). Using a number of approaches, cereal species such as rye (de la Pena et al., Nature 325:274-276 (1987)), corn (Rhodes et al., Science 240:204-207 (1988)), and rice (Shimamoto et al., Nature 338:274-276 (1989) by electroporation; Li et al. Plant Cell Rep. 12:250-255 (1993) by ballistic techniques) can be transformed.

Agrobacterium tumefaciens-meditated transformation techniques are well described in the scientific literature. See, for example Horsch et al. Science 233:496-498 (1984), and Fraley et al. Proc. Natl. Acad. Sci. USA 80:4803 (1983). Although Agrobacterium is useful primarily in dicots, certain monocots can be transformed by Agrobacterium. For instance, Agrobacterium transformation of rice is described by Hiei et al, Plant J. 6:271-282 (1994).

Transformed plant cells which are derived by any of the above transformation techniques can be cultured to regenerate a whole plant which possesses the transformed genotype and thus the desired RRK-controlled phenotype. Such regeneration techniques rely on manipulation of certain phytohormones in a tissue culture growth medium, typically relying on a biocide and/or herbicide marker which has been introduced together with the RRK nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The methods of the present invention are particularly useful for incorporating the RRK polynucleotides into transformed plants in ways and under circumstances which are not found naturally. In particular, the RRK polypeptides may be expressed at times or in quantities which are not characteristic of natural plants.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The effect of the modification of RRK gene expression can be measured by detection of increases or decreases in mRNA levels using, for instance, Northern blots. In addition, the phenotypic effects of gene expression can be detected by measuring lesion length as in plants. Suitable assays for determining resistance are described in U.S. Ser. No. 08/587,680.

The following Examples are offered by way of illustration, not limitation.

### EXAMPLE 1

As noted above, Xa21 genes make up a multigene family. Pulsed field gel electrophoresis and genetic analysis have demonstrated that most of the members of the Xa21 gene family are located in a 230 kb genomic region on chromosome 11 linked to at least 8 major resistance genes and 1 QTL for resistance (Song, et al., Science 270:1804 (1995); Ronald, et al., Mol. Gen. Genet. 236:113 (1992).

This example describes six Xa21 gene family members from the resistant rice line IRBB21, which members are designated A1, A2, C, D, E, and F. Cloning was as described in U.S. Ser. No. 08/587,680; Song, et al., supra and Wang, et al., Plant J. 7, 525 (1995). DNA sequences were determined by using the Sequitherm Long Read Cycle Sequencing Kit (Epicentre Technologies) in combination with the LI-COR Model 4000L Automated Sequencer (LI-COR Inc). To fill in gaps, a primer walking strategy was performed using synthesized primers (Operon) and the Applied Biosystems 373 DNA sequencer. Genebank accession numbers are as follows: A1: U72725 (SEQ ID NO: 4); A2: U72727 (SEQ ID NO: 10); C: U72723 (SEQ ID NO: 6); D: U72726 (SEQ ID NO: 1); E: U72724 (SEQ ID NO: 8); F: U72728 (SEQ ID NO: 12); 3′ flanking region of F: U72729 (SEQ ID NO: 12). The Wisconsin sequence analysis programs GAP and Pileup were used to calculate the percent identity and to carry out multiple alignments of DNA and protein sequences, respectively.

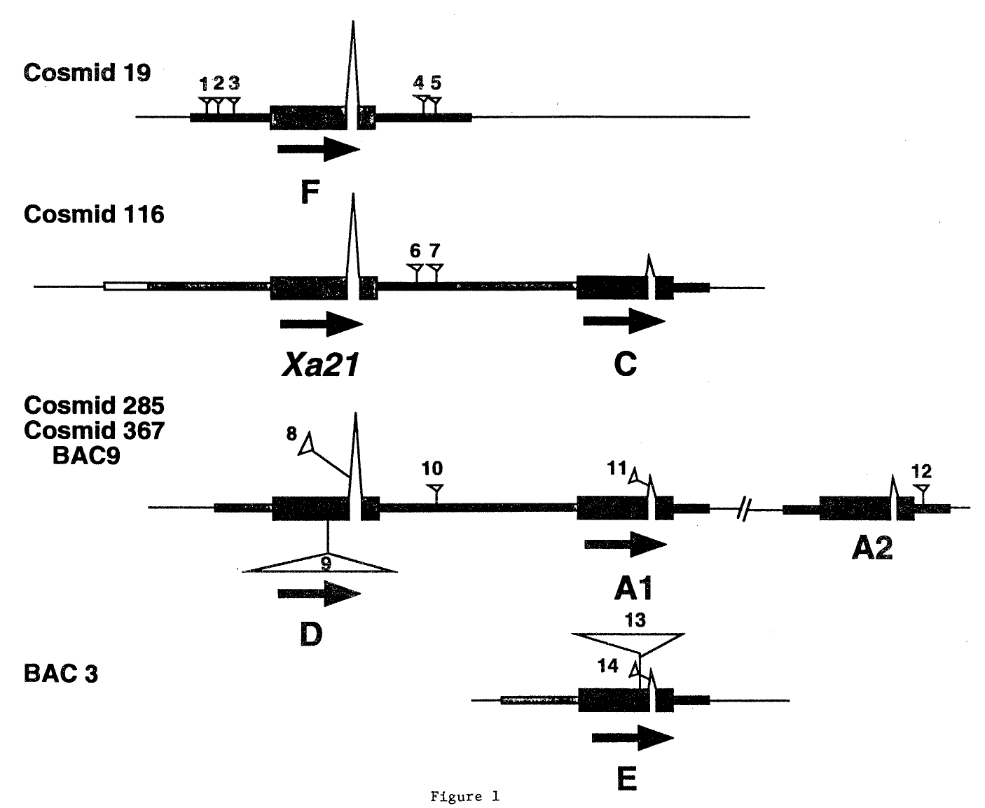

Sequence data and restriction enzyme analysis of cosmid and bacterial artificial chromosome clones indicated that the seven members are contained on 4 clones (FIG. 1). The first clone, carrying Xa21 (described in U.S. Ser. No. 08/587,680 and Song et al., supra. The Genbank accession number for Xa21 genmomic and cDNA sequences is U37133) and member C, spans a 40 kb region; the second clone includes member D, A1, and A2 and occupies a 150 kb region; clones of 40 kb and 130 kb contain members E and F, respectively. Genetic and molecular data suggests member E is inherited from the susceptible parent IR24 (P. C. Ronald, et al., Mol. Gen. Genet. 236, 113 (1992)).

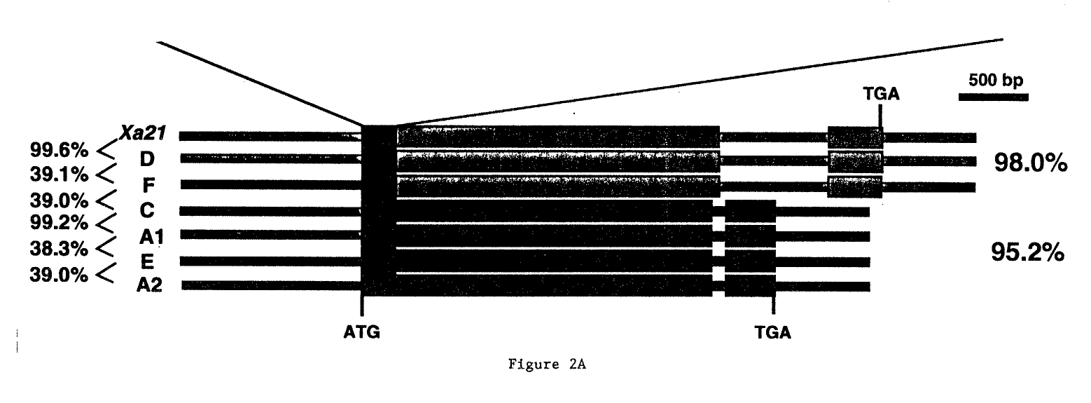

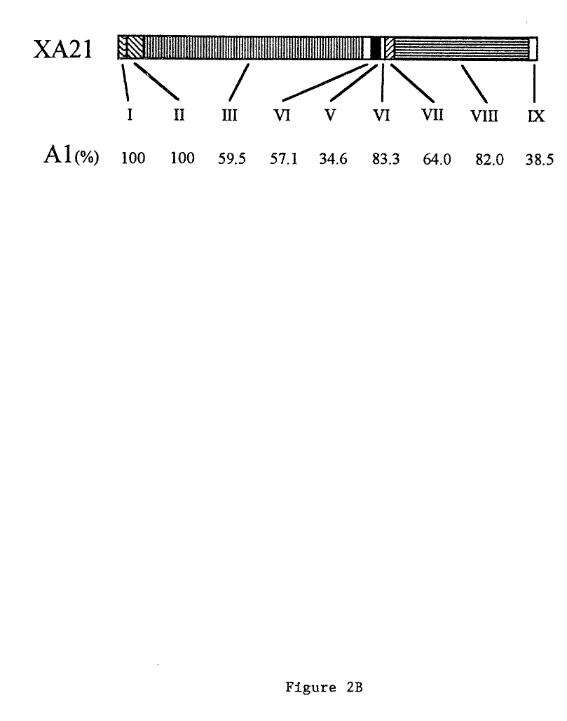

The entire coding region, the intron, and 3′ flanking region of the seven family members can be grouped into two classes. One class (designated the Xa21 class) contains Xa21, as well as members D and F (SEQ ID NOs: 1 and 12). The second class (designated the A2 class) contains members A1 (SEQ ID NO:4), A2 (SEQ ID NO:10), C (SEQ ID NO:6), and E (SEQ ID NO:8). Within each class, family members share striking nucleotide sequence identity (98.0% average identity for the members of the Xa21 class; 95.2% average identity for the members of the A2 class); compared to low levels of DNA sequence identity between members of the two classes (eg. 63.5% identity between Xa21 and A2) (FIG. 2A). Only the Xa21 and A1 open reading frames (ORFs) encode receptor kinase-like proteins. The sequence of other family members contain alterations causing a premature truncation of the predicted receptor kinase-like ORF (small deletions in F and C; base pair mutations in A2; or transposon insertions in D and E). At the amino acid level, A1 and XA21 share 68.6% identity overall. As shown in FIG. 2B, Domains I and II, carrying the presumed signal peptide and amino terminus of the protein, are 100% identical whereas the LRR domain (domain III) of XA21 and A1 share a low level of identity (59.5%) and differ in the number of LRRs (23 vs 22 respectively). In the presumed intracellular portion, the catalytic domains (domain VIII) of XA21 and A1 are highly conserved (82% identity), whereas the non-catalytic regions are divergent (64% identity for domain VII (juxtamembrane) and 38.5% identity for domain IX (carboxyl terminus)). The differences observed between members of the two classes suggest that they may differ in function. Indeed, we have found transgenic plants containing the A1 sequence are susceptible to all Xoo isolates tested.

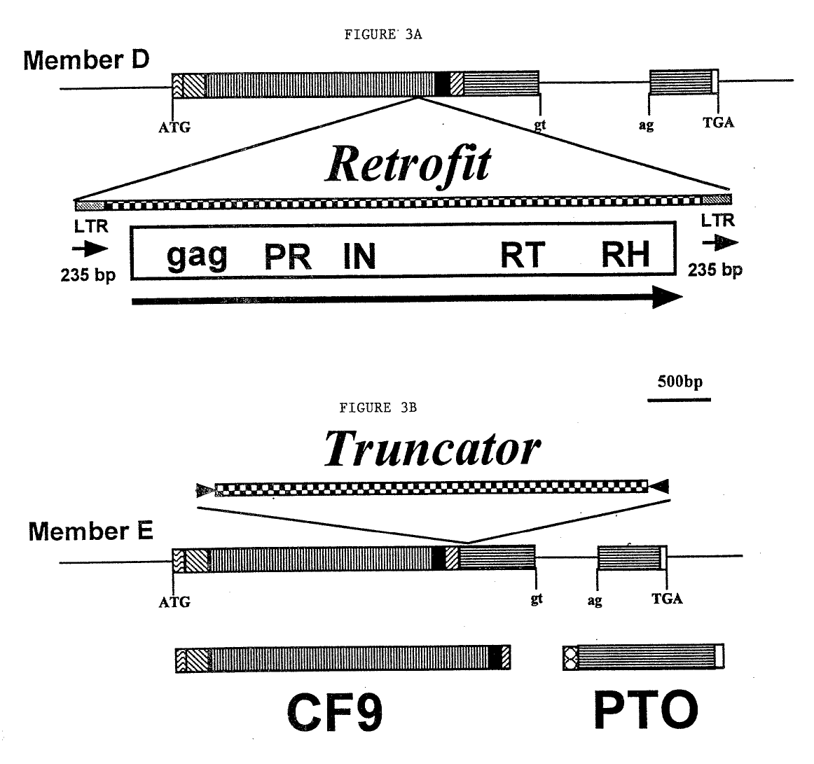

A remarkable feature of the Xa21 family members is the presence of fourteen transposable element-like sequences (M. A. Grandbastien, et al., Nature 337: 376 (1989); S. E.; White, et al., Proc. Natl. Acad. Sci. U.S.A. 91: 11792 (1994)). The position of these elements is shown in FIG. 1. Twelve elements insert into noncoding regions; whereas two elements, named Retrofit and Truncator, integrate into the coding regions of members D and E, respectively, resulting in disruption of the ORFs of these two members (FIG. 1, number 9 and 13). Retrofit (SEQ ID NO:3) belongs to the Drosophila copia class of retrotransposons and carries a large ORF showing greatest similarity to the ORF of maize Hopscotch (68.6% similarity; 54.6% identity) and tobacco Tnt1 (51.4% similarity; 31.9% identity) (M. A. Grandbastien, et al., Nature 337: 376 (1989); S. E.; White, et al., Proc. Natl. Acad. Sci. U.S.A. 91: 11792 (1994)). The insertion site of this element is located between the 23rd (V) and 24th (P) amino acids of the 22nd LRR creating a truncated molecule, lacking the transmembrane and kinase domains (FIG. 3A). Insertion of Retrofit into a presumed coding region contrasts with the observation in yeast and maize that integration of retrotransposons is biased towards noncoding regions (D. F. Voytas, Science 274: 737 (1996); P. SanMiguel, et al., Science 274: 765 (1996)). The fact that the truncated D confers partial resistance to Xoo suggests that transposition events at the Xa21 locus can alter expression of resistance.

Truncator, 2913 bp, represents a novel transposon-like sequence carrying 9 bp terminal inverted repeats (TIRs). The sequence shows no significant homology to any sequence in the database and contains no obvious ORFs. Interestingly, insertion of this element into the amino terminus of the kinase domain of member E would presumably result in premature truncation of the receptor kinase resulting in a receptor-like molecule structurally similar to the tomato fungal resistance gene products Cf9 and Cf2 (FIG. 3B) (D. A. Jones, et al., Science, 266: 789 (1994); M. S. Dixon, et al., Cell 84:451 (1996)).

In addition to the transposition events presented above, recombination between different family members was also found to play an important role in the evolution of the Xa21 locus. A 269 bp highly conserved (HC) region, located immediately downstream of the start codon of all seven family members marks the site of intragenic recombination events (FIG. 2A). The HC region, has a high G/C content (61.8% for Xa21) hallmarked by the typical G/C rich restriction enzyme recognition site Not I. At the amino acid level, the HC region spans domain I and domain H of XA21 and shares nearly 100% identity among seven family members.

The HC region delimits four classes of DNA sequences (˜1.3 kb) upstream of the HC region. The 5′ flanking region of family member F is divergent from that of other family members (less than 40% identity). The precise breakpoint (from sequence similarity to divergence) between Xa21 and F is located within the HC region, 120 bp downstream from the start codon. This sudden change of sequence identity is unlikely due to random events such as transposon insertion or deletion because such events would presumably lead to an altered coding region. This is not the case; the deduced amino acid sequence of F maintains the receptor kinase like ORF. These results suggest that a recombination event occurred in the HC region resulting in the formation of a chimeric sequence containing the 5′ flanking region of F and a downstream region (including coding region, intron, and 3′ flanking region) of the Xa21 class.

In further support of the idea that the HC region mediates intragenic recombination, we also observed apparent recombination breakpoints near or within the HC region for gene family members E, A1, and C. For E, the 5′ flanking region is divergent from all other members whereas the 3′ downstream regions belong to the A2 class. The sudden change of DNA identity can be explained by a recombination event between a progenitor A2-type gene and an unknown family member. The likely recombination breakpoint in E is located 105 bp upstream of the HC region since sequences upstream of this site are quite different, compared with a high level of DNA sequence identity downstream of this site.

The nearly identical DNA sequences of C and A1 provide the most striking example of an HC mediated recombination event. For example, the 5′ flanking region of C shows nearly perfect identity (99.2%) to that of Xa21, whereas the downstream region of C belongs to the A2 class. The high level of identity between the 5′ flanking sequences of Xa21 and C extends 3.8 kb upstream. This upstream region includes the functional promoter for the Xa21 gene (W.-Y. Song, et al., Science 270:1804 (1995)). These results strongly suggest that C was created by a recombination event in the HC region between progenitors of the Xa21 and A2 classes. The likely recombination breakpoint in member C is delimited by two characteristic deletions: one is located at position −37 and is only present in Xa21 class members (Xa21, D, C, and A1); another deletion is located at position 255 and occurs in all A2 class members.

From these results it is clear that we have identified a highly conserved, G/C rich region in the gene family and that this region appears to be involved in high frequency recombination between family members. Not only is the HC region present in O. longistaminata, but is also present in Xa21 family members of the cultivated rice species O. sativa (The clone RG103, spanning the HC region of an Xa21 gene family member was isolated from O. sativa cultivar IR36 (3, S. Mcouch, et al., Theoret. Appl. Genet. 76:815 (1988)). Genebank accession number of RG103 is U82168. The mechanism for HC region-mediated recombination is unknown; however, two models can be envisioned. First, this region may mediate programmed recombination similar to that observed in African trypanosomes (R. H. A. Plasterk, Trends Genet 8, 403 (1992)). In trypanosomes, antigenic variation is controlled by a variant surface glycoprotein (VSG), which is encoded by a member of a multigene family containing more than 1000 members. Recombination at stretches of highly conserved nucleotides between silent and expressed members of the VSG gene family leads to expression of new antigens. Alternatively, HC mediated recombination may be an example of an ectopic recombination event where the HC region serves as a recombination initiation site (T. D. Petes, et al., Annu. Rev. Genet. 22:147 (1988); A. Nicolas, et al., Nature 338: 35 (1989)). Frequent recombination in this region would maintain the conservation of the HC region but allow flanking sequences to diverge. Over time, mismatch repair would lead to homogenization of the HC region and result in an overall increased G/C content as has been observed in yeast (Brown T., et al., Cell 54, 705 (1988)).

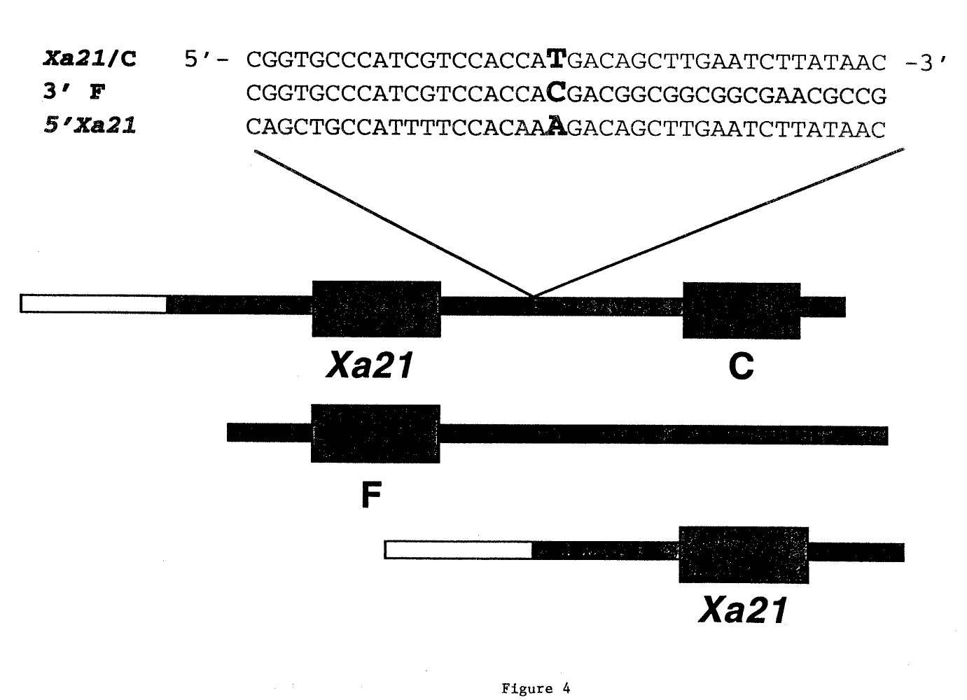

Evidence for recombination in intergenic regions of the Xa21 family members was also observed. First, sequences in the 5′ flanking region of members C and Xa21 are identical for 3.8 kb and then abruptly diverge. Interestingly, the same site of divergence is observed in the 3′ flanking regions of Xa21 and member F (FIG. 4). The presence of a conserved site of divergence suggests not only that this is a recombination breakpoint but that the Xa21/C cluster and member F are generated from the same progenitor. Second, the sequence of a 14742 bp region spanning the Xa21/C cluster shows 97.7% identity to the corresponding sequence (14871 bp) of the D/A1/A2 cluster (FIG. 1), suggesting these regions evolved through sequence duplication. This duplication process can be explained by a presumed unequal cross-over event in the intergenic region of these two clusters.

### EXAMPLE 2

Using PCR amplification techniques as described in U.S. Ser. No. 08/587,680, Xa21 genes were isolated from cassava (SEQ ID NOS: 13-14), maize (SEQ ID NO: 15-16) and tomato (SEQ ID. NOs: 17-29). The following is a description of the methods used to isolate TRK1-7 from tomato. The same general procedure was used for maize and cassava.

We designed primers in conserved regions of both the Leucine Rich Repeat (LRR) region and the serine-threonine kinase domain of Xa21. The PCR products should amplify between these two domains and therefore span the transmembrane domain. So far, two sets of primers have proven successful to amplify three homologues of Xa21 in tomato.

The first clone TRK1 is a cDNA and the encoded polypeptide (SEQ ID NOs:17and 18). This clone is present as one or two copies in the tomato genome and one copy maps to the short arm of chromosome 1 in the proximity of a resistance gene to Xanthomonas campestris pv. vesicatoria (Rx1)(Zu et al. (1995) Genetics 41:675-682).

The second clone TRK2 (SEQ ID NO:19) is a 496 bp PCR product with an ORF encoding a polypeptide (SEQ ID NO:20). TRK2 maps within a few cM of mcn (FIG. 4) a mutation on chromosome 3 that mimics disease lesions. A third clone TRK3 (SEQ ID Nos: 21 and 22) is a 473 bp fragment and maps to chromosome 8 near an erecta like mutant. TRK4-7 (SEQ ID Nos: 23-29) are further PCR products and encoded polypeptides

Primers that have been proven useful are as follows.

1. LRR region

L3a. TCA AGC AAC AAT TTG TCA GGN CA(A/G) AT(A/C/T) CC

2. Kinase region

K1a. CGC CTT AGG ATT TTC AAG CTT TCC (T/C)TT (G/A)TA NAC
K2a. TAA CAG CAC ATT GCT TGA TTT NAN (G/A)TC NCG (G/A)TG
K2b. TAA CAG CAC ATT GCT TGA TIT NAN (G/A)TC (G/A)CA (G/A)TG
K2c. TAA CAG CAC ATT GCT TGA TTT NAN (G/A)TC (T/C)CT (G/A)TG

The following combinations of primers are preferred:

L3a+K1a then L3u|K1u

L3a|K2a then L3u|K2u

L3a+K2b then L3u+K2u or

L3a+K2c then L3u+K2u.

PCR conditions

first cycle

94 for 30 s

55 for 30 s

72 for 1 min

For the next 19 cycles, the annealing temperature drops 1 degree C. every cycle. After 20 cyles, 10 min at 72. After inital amplification as second round of amplification is performed with the following specific primers with 1 microliter of the previous PCR.

L3u. TCA AGC AAC AAT TTG TCA
K1u. CGC CTT AGG ATT TTC AAG CTT
K2u. TAA CAG CAC ATT GCT TGA

The conditions for this amplification are:

35 cycles

94 15 sec

55 15 s

72 1 mn

after 35 cyles, 72 for 10 min

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

What is claimed is:

1. An isolated nucleic acid construct comprising an RRK polynucleotide sequence, which polynucleotide hybridizes to SEQ ID NOs: 1, 4, 6, 8, 10, or 11 under stringent conditions.

2. The nucleic acid construct of claim 1, wherein the RRK polynucleotide sequence encodes an RRK polypeptide having an leucine rich repeat motif.

3. The nucleic acid construct of claim 1, wherein the RRK polynucleotide sequence encodes an RRK polypeptide having a cytoplasmic protein kinase domain.

4. The nucleic acid construct of claim 1, wherein the polynucleotide sequence is a full length gene.

5. The nucleic acid construct of claim 1, wherein the Xa21 polynucleotide is as shown in SEQ ID NOs: 1, 4, 6, 8, 10, or 11.

6. The nucleic acid construct of claim 1, further comprising a promoter operably linked to the RRK polynucleotide sequence.

7. The nucleic acid construct of claim 1, wherein the promoter is a tissue-specific promoter.

8. The nucleic acid construct of claim 1, wherein the promoter is a constitutive promoter.

9. An isolated nucleic acid construct comprising a cassava RRK polynucleotide sequence, which polynucleotide hybridizes to SEQ ID NO: 13 under stringent conditions.

10. The isolated nucleic acid construct of claim 9, which is SEQ ID NO: 13.

11. An isolated nucleic acid construct comprising a maize RRK polynucleotide sequence, which polynucleotide hybridizes to SEQ ID NOs: 15 or 16 under stringent conditions.

12. The isolated nucleic acid construct of claim 11, which is SEQ ID NO: 15 or SEQ ID NO: 16.

13. An isolated nucleic acid construct comprising a tomato RRK polynucleotide sequence, which polynucleotide hybridizes to SEQ ID NOs: 17, 19, or 21 under stringent conditions.

14. The isolated nucleic acid construct of claim 13, which is SEQ ID NO: 17, SEQ ID NO:19, or SEQ ID NO:21.

15. A transgenic plant comprising a recombinant expression cassette comprising a plant promoter operably linked to a Xa21 polynucleotide sequence of claim 1.

16. A method of enhancing resistance to Xanthomonas in a plant, the method comprising introducing into the plant a recombinant expression cassette comprising a plant promoter operably linked to an RRK polynucleotide sequence of claim 1.

17. The method of claim 16, wherein the plant tissue is from rice.

18. The method of claim 16, wherein the plant tissue is from tomato.


SEQUENCE LISTING (53 sequences)

SEQ ID NO: 1 - DNA (genomic), 13341 bp, Oryza longistaminata; single-stranded, linear
  CDS 2367..4205: /product= “receptor kinase-like protein” /note= “Xa21 gene family member D”
  misc_feature 4201..9071: /note= “retrofit, a copia-like, transposon-like element”
  CDS 4484..8821: /product= “retrofit” /gene= “gag/pol”
  intron 9915..11712
  misc_feature 10020..10975: /note= “Krispie, transposon-like element”
  misc_feature 12626..12750: /note= “Pop-O12, transposon-like element”
  misc_feature 13040..13248: /note= “Ds-rice2, transposon-like element”
  original source strain: IRBB21
  chromosome/segment: 11
  map position: 11q, RG103
aagcttcattggtttcttcagttatacttacgtaggtttttcctgtatacataaatacgtaacagagtaagggaattagattgtttaaaataaaatacatataatctaatagcctaaaatatcaggtccactgacagtggcggatctaggatttagaatatgggtggtccgacctaattttttcctaaacatactaaatctaacgatggtaatatatactatgcaagtatagataatagaatagaccaaaagtgtatcatgctatattaataaagcatcttaaaacatatataattaataattacctaaaattttgacttaaagaagctcacatggctataaaagtttaaagaaaattaccatactaatttttcttcttatcgggtctacgccttctaatggccatgaaagtggtcgttatatcttcttccttcactcttaagaaaacatcccgcttaatggatgtgtctatactatcatccaaaagctcatcacccatcttttttctcaaccatcattagtaaatgcatcagttctactataatttaatatcacaatgcacaggagtaaagagttcaaaatttcaaaactgaaaattgaaaaaaaagtaaaaaaaaaatagaaaacctttttgttttggcttggtgcaggtctgcaccagtgcgctagtgcggcactgcggcggcagcggccaaggtgtcgacgcgcgtgcgtggcccggtggcgctcgctctcacgatctgatcagatcgctgatcgcgtcggcgtcgcgactcgcgagggcgaggaggagagcgacagagagtcctgcgacggcgcgacgcttcggtttcttaattccgaacgattagatacaccgtacacgcgcgtgtggtgtggggcctgtggtaatctaatggtttaaaatattgggtccaccaatttaagtgaaaatcgacggttagatatgatagagctacgtggcagcctaagagcgtttgtaggagtcccacgtggcggtttgagagcgtttgtaggaagtttaatggacttttagtatataatagatatttataattttattaagtaccctaattttccctaaacaatttttctctctcatcgtatttccatatatctttttgagataataatggatataaacatagctagaaatgtaaatgttcaccttgcatcaataggggatgaagttgctaaccttttagatctcctcgatttgtataatataaccaaaatattttcaccaaaaatttcgttaaacatccgagatatttgttgtttttgccgatcgagcaaagattagtagtccagcagtgtctgcaccaccaccatcgtgataatgcatcttgtgtgttattcttgatgagaaaatacgtagtgaaaaccacatatgtggtggaaacttagaaactaccgttagatcgagaaatggatgtccaagattcgtccacgtcaccaagagataaaatttaactcgcagattcacttatgagttaaaattttaatgagagttaaattttaactcatgttgatgtggacgaatatcggacatccatttctcgatccaacgatagcttccaagtttccactacatatgtggtttgcactatatattttcccattcttgattatgtgtttgagagcagctagcacaaagagaaaaaaaagcatcgtttttcacgcgtatgttttcagaactgttaaatggtgtgttttttgaaaaaactttctatagaaaagtttctttaaaaaatatattaatctattttttaagtttaaaataattactacttaattaattatacactaacagcttatttcgttctacgtatcttgtcaattttcgctattcctttcttctcaaacacggcattggatgctctcatagcacttgctcgttcggatagaagacttgacgaagacgaccgctacaacttggtgtgttatatcgtgctttgtttagcataatcattacatatattccatgccgaagtgccgacgatgagaccgtgttcgatgcatctttgtatggcatctagggacaaagagcatagagtccctaccatagtacctgctcgcgtagaagacttgacgagaagaccgactgctacaccttggtgtgtaataatatcgtgttgtgtgtaccatgcatactcctttaaaacaaataatggtggtaacagtaaatctgtcatcccacccactctcattgtaaattttgcaagttatcacttgaacttcttaatactccatccgtttgcgtgtgttctttcagaatttgcgtgagcactttttcttctatataatctgtctagtccatgagctaaaccagcatctctcgctgtcttgccttgcacttctgcacgatgatatcactcccattattgctcttcgtcctgttgttctctgcgctgctgctctgcccttcaagcagtgacgacgatggtgatgctgccggcgacgaactcgcgctgctctctttcaagtcatccctgctataccaggggggccagtcgctggcatcttggaacacgtccggccacggccagcactgcacatgggtgggtgttgtgtgcggccgccgccgccgccggcacccacacagggtggtgaagctgctgctgcgctcctccaacctgtccgggatcatctcgccgtcgctcggcaacctgtccttcctcagggagctggacctcggcgacaactacttctccggcgagataccaccggagctctgccgtctcagcaggcttcagctgctggagctgagcgataactccatccaagggagcatccccgcggccattggagcatgcaccaagttgacatcgctagacctcagccacaaccaactgcgaggtatgatcccacgtgagattggtgccagcttgaaacatctctcgaatttgtaccttcacaaaaatggtttgtcaggagagattccatccgctttgggcaatctcactagcctccaggagtttgatttgagcttcaacagattatcaggagctataccttcatcactggggcagctcagcagtctattgaatatgaatttgggacagaacaatctaagtgggatgatccccaattctatctggaacctttcgtctctaagagcgttttgtgtcagcgaaaacaagctaggtggtatgatccctacaaatgcattcaaaacccttcacctcctcgaggtgatatatatgggcactaaccgtttccatggcaaaatccctgcctcagttgctaatgcttctcatctgacacggcttcagattgatggcaacttgttcagtggaattatcacctcggggtttggaaggttaagaaatctcacagaactgtatctctggagaaatttgtttcaaactagagaacaagaagattgggggttcatttctgacctaacaaattgctccaaattacaaacattgaacttgggagaaaataacctggggggagttcttcctaattcgttttccaatctttccacttcgcttagttttcttgcacttcatttgaataagatcacaggaagcattccgaaggatattggcaatcttattggcttacaacatctctatctctgcaacaacaatttcagagggtctcttccatcatcgttgggcaggcttaaaaacttaggcattctactcgcctacgaaaacaacttgagcggttcgatcccgttggccataggaaatcttactgaacttaatatcttactgctcggcaccaacaaattcagtggttggataccatacacactctcaaacctcacaaacttgttgtcattaggcctttcaactaataaccttagtggtccaatacccagtgaattattcaatattcaaacactatcaataatgatcaatgtatcaaaaaataacttggagggatcaataccacaagaaatagggcatctcaaaaatctagtagaatttcatgcagaatcgaatagattatcaggtaaaatccctaacacgcttggtgattgccagctcttacggcatctttatctgcaaaataatttgttatctggtagcatcccatcagccttgggtcagctgaaaggtctcgaaactcttgatctctcaagcaacaatttgtcaggccagatacccacatccttagcagatattactatgcttcattccttgaacctttctttcaacagctttgtgggggaagtgccaaccatgtgaaacagtctgttctattatcctggacatgagatatacatgcatgctccagtacatgcgatgctctagtactgggatatggttcgttaggataaggagattatctccaaatttgtttcaccttcttatccattgtaactgaatactgtaagccacggtagaggctgtttctaccactatttaacagagatgcggcccaaggcaaagggtttaacgcttcacattttatcacatggtatcagagcctttttccacctcaatagatcgcatctttcatccacatggcgtcgtcgtcgtcttcctcaggcgcggcagccgccaatctcctccaaggccactcggtttcagagaaactcgggaaagccaaccatgcattgtggaaagcgcaagttagcgctgcagtgcgtggagcccgattgctgggctacctcaacggcgatatcaaagctccagacgccgaactctcggtcaccatagatgggaagaccacaacaaagccgaatccggcatttgaagattgggaggccaatgaccagcttgttcttggctatctcctgtcatctctttcaagggatgtgctgatccaagtcgccacatgcaagacggcggctgaggcatggcggagcattgaagcactctactccaccggcactcgagcaagggcggtgaacaccagactcgccctcaccaacacgaagaaaggaacaatgaagatcgccgagtatgtcgccaagatgcgagcgcttggtgatgagatggctgccggcggtcatccacttgatgaagaagaccttgtccagtacatcatcgctgggctaaatgaagacttcagcccgatcgtctccaacctctgcaacaagtccgatcccatcacggttggggagctgtattctcagctcgtcaactttgaaaccctccttgatctctaccgcagcactggtcagggaggagctgcttttgtcgctaatcgcggcaggggcggcggcggcggcgggcgcggcaacaacaacaactccggcggcggcggcggcagaagcgcgccgggtggacgcggcagcggcagccagggtcgcggtggccgtggacgcggcacaggaggccaagacaggcgccctacttgccaagtttgtttcaagcgtgggcatacagcagctgattgttggtatcgcttcgacgaggactacgttgcagatgagaagctcgttgctgctgctactaactcgtatggtatagatacaaattggtatattgatacaggtgctacagatcacattaccggtgaactagagaagcttaccaccaaggagaaatacaacggcggcgagcaaattcacactgctagcggagcaggtatggatattagtcacattggtcatactattgtgcatacccctagccgtaatattcatctaaataatgtcctttatgttcctcaagccaagaaaaatcttatatctgctagtcaattagccgctgataattctgcttttcttgaacttcactcgaaattcttttctataaaggatcaggtaacgagggacgttctgcttgaagggaaatgtagacacggtctctacccgatccccaagttctttggtcgctcaaccaacaaacaagcccttggtgccgccaagttatccctgtctaggtggcatagccgtctaggacatccgtctcttcctattgttaagcaagtcattagcagaaataatctcccatgttcagttgagtcagtcaatcagtctgtgtgtaatgcttgccaagaagcaaagagtcatcagttaccttatattagatctactagtgtgtctcaatttcctcttgaacttgttttttctgatgtttggggccctgctccagagtctgttgggagaaataaatattatgtgagtttcattgatgattttagtaagtttacttggatatacttgctgaaatacaagtctgaggtttttgagaaatttaaagaatttcaggctttagttgaacgaatgtttgatagaaagattattgccatgcagactgattggcggggggggagatatcagaaacttaattccttttttgctcaaataggattgatcatcatgtgtcatgtcctcacactcatcaggcagaatgggtcagctgagagaaaacaccggcatatcgtggaagtaggcctttctcttttatcttatgcatcaatgcctcttaagttttgggatgaagcctttgttgcagccacttatctcatcaatcgtatacctagtaaaaccatccaaaattctacacccctagagaaactgtttaaccaaaaacctgactactcatccttgagagtgtttggttgtgcatgttggcctcatcttcgcccttacaatacacacaaactccagtttcgctccaaacagtgcgtgtttttgggttttagtactcaccacaaaggatttaagtgtcttgatgtgtcatcaggccgtgtctacatctcaagagatgttgtctttgatgaaaatgtttttcccttctctacactccactcaaatgcaggagccagactcaggtctgaaattcttttgttgccgtcccccttgacaaactataatacggctagtgcagggggaacacatgtagttgcaccagtggctaatactccattacctagtgataatttaatttctaatgctgctgatgtgacttctggagaaaatagtgcagcacatgaacaggaaatggagaatgagcaggaaatagagaacgtcatgcatgggaacgacgtgcatggggacgcggcatcgggacctgtgctggatcaaccaactgctgacagcagcactgcgccggaccagggagctgacaccagtgacgcggtctctggcgcagcttctgacgcgggtggagacactgccaccctgggagctggagcagcaaatagcgcagcagcaggtggtgaagaatcccagccggtgcagcctgatgtgacgggtacagtactggctacagtagcccctgcatcgagaccacacactcgtctgcggagtggtattcgaaaagagaaggtatacactgatggcaccgttaaatatggttgtttttcttctactggtgaaccacaaaatgataaagaggctttaggagataaaaactggagagatgcaatggaaactgagtataatgctttgataaaaaatgacacatggcacctagttccatatgagaaaggacaaaatatcattgggtgtaaatgggtatataagattaaaaggaaggcagatgggacacttgatagatacaaagctagacttgtagcaaaggggtttaaacaaagatatggtatcgattatgaagatacttttagtcctgttgttaaagctgctactattagaattattctgtccattgctgtctctagaggttggagtcttagacagttagatgttcagaatgcctttcttcatggattcttagaagaagaagtctacatgcaacaacctcctgggtttgagtcatcctctaaacctgattatgtatgtaaattggataaggcattatatgggctgaaacaagcaccaagggcgtggtattccaggctgagtaagaaacttgttgaacttggttttgaagcttcaaaggctgatacctcattattctttcttaacaaaggagggatacttatgtttgttttggtatatgttgatgatataattgtagctagctctacagagaaggcaactacagcacttctgaaggatctaaacaaggagttcgcacttaaggatttgggagacctgcactacttccttggaattgaggtaactaaagtttccaatggcgttatcttgactcaagagaagtatgcaaatgatctgctaaagagagttaatatgtcaaattgcaagccagttagtactcctctttctgttagtgaaaaattaactctatatgagggatcacccttgggtcctaatgatgcaatacaatatagaagtatagttggtgctttacaatacttgaccttgacaagacctgacatagcttattcagtaaacaaagtctgtcagtttcttcatgctcctactaccagtcattggattgcagtaaaaagaatcctcagatacttgaaccaatgcacaagtctaggacttcatatacacaagagtgcttctactcttgttcatgggtattctgatgcagactgggcaggtagtatagatgacagaaaatcaacaggaggatttgcagtatttttgggttctaatcttgtgtcctggagtgctaggaaacaacctactgtgtcaaggtcaagcacagaggcagaatataaggctgtggcaaatactacagccgaactgatatgggtacaaaccttgttaaaagaattgggaattgagtctcctaaagctgccaagatttggtgtgataacttaggagctaaatatttatcagctaatcctgtgtttcatgcaaggacaaagcatatagaggttgattatcattttgtaagagaacgagtgtcacagaagctgttagagattgattttgttccatcaggagaccaagttgctgacgggtttacaaaggcactgtcagcttgtcttcttgaaaattttaaacacaatcttaacctagctaggttatgattgagagggctgtgaaacagtctgttctattatcctggacatgagatatacgtgcatgctccagtacatgcgatgctccagtactgggatatggttcgttaggataaggagattatctccaaatttgtttcaccttcttatccattgtaactgaatactgtaagccacggtagaggctgtttctaccactatttaacagagatgcggcccgaggcaaagggtttaacgcttcacattttatcacaaaccattggtgctttcgcagctgcatccgggatctcaatccaaggcaatgccaaactctgtggtggaatacctgatctacatctgcctcgatgttgtccattactagagaacagaaaacatttcccagttctacctatttctgtttctctggtcgcagcactggccatcctctcatcactctacttgcttataacctggcacaagagaactaaaaagggagccccttcaagaacttccatgaaaggccacccattggtctcttattcgcagttggtaaaagcaacagatggtttcgcgccgaccaatttgttgggttctggatcatttggctcagtatacaaaggaaagcttaatatccaagatcatgttgcagtgaaggtactaaagcttgaaaatcctaaggcgctcaagagtttcactgccgaatgtgaagcactacgaaatatgcgacatcgaaatcttgtcaagatagttacaatttgctcgagcattgataacagagggaacgatttcaaagcaattgtgtatgacttcatgcccaacggcagtctggaagattggatacaccctgaaacaaatgatcaagcagaccagaggcacttgaatctgcatcgaagagtgaccatactacttgatgttgcctgcgcactggactatcttcaccgccatggccctgaacctgttgtacactgtgatattaaatcaagcaatgtgctgttagattctgatatggtagcccatgttggagattttgggcttgcaagaatacttgttgatgggacctcattgatacaacagtcaacaagctcgatgggatttagagggacaattggctatgcagcaccaggtcagcaagtccttccagtattttgcattttctgatctctagtgctatatgaaatagtttttacctctagtgaaactgatggagaatataagtaattaattgaactcaggggcgaagcagaataaatcatcgccggggtcactactaactaatgaacttgcactactatgaacaggtttatcgtaaatggccaaaacacatttttacaggtggccaatcgtgtcgcttatgactaaagatcttttcgcttgtgaccggtgctacgataatcacatggtgaaatgaatcttcacagatatagcctttaagtttaagctagccatttgcagaaaatgaaaggggtggctgtcagaaaaaaaaaaccatttttttgtagtgtgactaaaactatgcgtaagatggaacaaaattatacattaatcctcctccttactcatgatcatataactgaagtttgaaaacaaagacaaatcaaatgttcatatttcagaacttctgaattatagaaccctaacttcctaataaactttcggacttgagaaaccaccctagggctaaattgtgatggtaataatcaagaaattgtgataatgttagtaggttaccgccctcaacagctcgagcgctcgagcgctcaaccataacccttcacccttgccattcttctcgtccttgttacgccaagccggcgatgcacgcgtgtactactcgaggacgataagacgcggaaagtcgagaaatcgagatgtgcggcgatgcaatgcgaagtgctcggtcaaactaccagaagaatatgatgcaacaggggaatggactttctgggctgccggccatgtgggctcaagtctcaggcgtttaagagctgattaattttctattttctacttactagtctcgttcctaattgctattgggctttaacaaaacggtgtcaccggggtccagtagtttgcttgactggggtatagctagtaaaaaagcgaggggtactagatgtatgcacgaaatcgtcggggtctactgaccccgatggtaggctgtagcgtcgcccctgattgaactaattaaattgcacaaaaataagattatttgccatatctattcagatgctaaatatagctagttcatagaggtacagatttttttatataggactctagagctaccacacactcaaatcaaattatgggtgtgttctgctctacactgcaatatgaaatgattattacttctacatgaactgatggaggagtttcagaaggatcaaatttgagtaaaattttcaattctacatttaagaaacacttttttttcatatgctagttacaattttttatttcacgagcttacattgaccatgaaaaatacttggcactacttactaattcccacatggaggtagtgaaaataatatagatacaaaaacgaaatatcctatgttgtgtgatatactataatcacaatgaacacaaacaggattcgtacaaaagtaattagccatcatagcaactgattgcttggggtaactgtatagcacaatcataccaaatttctttagatatgtatctgtaaattagattcttaaagttaaatatgaaatttcattggtatttatgtttctttatataataaaaattaatccagcctttgcatctatcatttgtccagacatccttgttatttgtgatatttaacacgtaaatttacataattatacatccaagttctttttatttaacactgtaaatttcaaatcgtacatgttataaagaatgtactatatttcctgctcaaacagagtatggcgttgggctcattgcatcaacgcatggagatatttacagctatggaattctagtgctggaaatagtaaccgggaagcggccaactgacagtacattcagacccgatttgggcctccgtcaatacgttgaactgggcctacatggcagagtgacggatgttgttgacacgaagctcattttggattctgagaactggctgaacagtacaaataattctccatgtagaagaatcactgaatgcattgtttggctgcttagacttgggttgtcttgctctcaggaattgccatcgagtagaacgccaaccggagatatcatcgacgaactgaatgccatcaaacagaatctctccggattgtttccagtgtgtgaaggtgggagccttgaattctgatgttatgtctcgtaatgttttattgccactcttcagatcgacttctgcagtggtatctaccacacgatcactaaagtcaccgtggttatttcctgatccagcatatctgatcatgcatgttctgtgttgtatacctgtattttactctgaattgccacaccgcaaccctgcatctgtttgtttggtatacaaaagatagtgatgagtttattgttttaggggcttcctagttggcgcgtgtggtgccggcacgcacgcagcccgagggtgggtttcttttttttccattgttattccgttgctttttttcaccacggtagatcttttttttccggatttccattttttccgttgtttttctctatcgcttatgttggcggatttttttccgtggttttctttccgaagacgagtatatctaacgtaactaacatgttacttttagataacgatggttattaagataagatttttctctggaagatttttgtaagtaacagattgaaaacaaatctatacgtgaggtcaaattttgaaaactttcaatctagatttaaaaacttttcaactcaaaatttgaatttttgaagtgaaaatttgaaaactttcaaaaattactagtaatcgacaaaaaaaaaatggaaatggaaacggaaatagttttgctgttataccgatcgtttccatatttaccgtattcttatagaaattaccgtttcttataatatggtaattaccgtatttctaaatatgttgatatttatagggcatgtctctacttgactcacagtttagagattgattgactatttaatcaaatccctaacttgattgcatggctaaaatggagttgatttctaatttatatagtatagcttgaatttatttgtaaatataacatacttatgtaaagttaaatatatgttttctatagtttaatgtttctgtatttgttaccggttttcgatctgtaccgacatgtttccatcagtattattccatgtccggttttccgatatttccgatatcgttttcgtttccgactttaccgttttcgatttcatttccgagaaaaatatgattatggaaatggtcgaggctgttttccgatcgtttccgaccgttttcatctctacccgtagtaataatatataacattttatctctaatctttctctctctcatatcaatgaaataatcgctaagagactgctattaacaagggctt

SEQ ID NO: 2 - protein, 612 aa
MISLPLLLFVLLFSALLLCPSSSDDDGDAAGDELALLSFKSSLLYQGGQSLASWNTSGHGQHCTWVGVVCGRRRRRHPHRVVKLLLRSSNLSGIISPSLGNLSFLRELDLGDNYFSGEIPPELCRLSRLQLLELSDNSIQGSIPAAIGACTKLTSLDLSHNQLRGMIPREIGASLKHLSNLYLHKNGLSGEIPSALGNLTSLQEFDLSFNRLSGAIPSSLGQLSSLLNMNLGQNNLSGMIPNSIWNLSSLRAFCVSENKLGGMIPTNAFKTLHLLEVIYMGTNRFHGKIPASVANASHLTRLQIDGNLFSGIITSGFGRLRNLTELYLWRNLFQTREQEDWGFISDLTNCSKLQTLNLGENNLGGVLPNSFSNLSTSLSFLALHLNKITGSIPKDIGNLIGLQHLYLCNNNFRGSLPSSLGRLKNLGILLAYENNLSGSIPLAIGNLTELNILLLGTNKFSGWIPYTLSNLTNLLSLGLSTNNLSGPIPSELFNIQTLSIMINVSKNNLEGSIPQEIGHLKNLVEFHAESNRLSGKIPNTLGDCQLLRHLYLQNNLLSGSIPSALGQLKGLETLDLSSNNLSGQIPTSLADITMLHSLNLSFNSFVGEVPTM

SEQ ID NO: 3 - protein, 1445 aa
MASSSSSSGAAAANLLQGHSVSEKLGKANHALWKAQVSAAVRGARLLGYLNGDIKAPDAELSVTIDGKTTTKPNPAFEDWEANDQLVLGYLLSSLSRDVLIQVATCKTAAEAWRSIEALYSTGTRARAVNTRLALTNTKKGTMKIAEYVAKMRALGDEMAAGGHPLDEEDLVQYIIAGLNEDFSPIVSNLCNKSDPITVGELYSQLVNFETLLDLYRSTGQGGAAFVANRGRGGGGGGRGNNNNSGGGGGRSAPGGRGSGSQGRGGRGRGTGGQDRRPTCQVCFKRGHTAADCWYRFDEDYVADEKLVAAATNSYGIDTNWYIDTGATDHITGELEKLTTKEKYNGGEQIHTASGAGMDISHIGHTIVHTPSRNIHLNNVLYVPQAKKNLISASQLAADNSAFLELHSKFFSIKDQVTRDVLLEGKCRHGLYPIPKFFGRSTNKQALGAAKLSLSRWHSRLGHPSLPIVKQVISRNNLPCSVESVNQSVCNACQEAKSHQLPYIRSTSVSQFPLELVFSDVWGPAPESVGRNKYYVSFIDDFSKFTWIYLLKYKSEVFEKFKEFQALVERMFDRKIIAMQTDWRGGRYQKLNSFFAQIGLIIMCHVLTLIRQNGSAERKHRHIVEVGLSLLSYASMPLKFWDEAFVAATYLINRIPSKTIQNSTPLEKLFNQKPDYSSLRVFGCACWPHLRPYNTHKLQFRSKQCVFLGFSTHHKGFKCLDVSSGRVYISRDVVFDENVFPFSTLHSNAGARLRSEILLLPSPLTNYNTASAGGTHVVAPVANTPLPSDNLISNAADVTSGENSAAHEQEMENEQEIENVMHGNDVHGDAASGPVLDQPTADSSTAPDQGADTSDAVSGAASDAGGDTATLGAGAANSAAAGGEESQPVQPDVTGTVLATVAPASRPHTRLRSGIRKEKVYTDGTVKYGCFSSTGEPQNDKEALGDKNWRDAMETEYNALIKNDTWHLVPYEKGQNIIGCKWVYKIKRKADGTLDRYKARLVAKGFKQRYGIDYEDTFSPVVKAATIRIILSIAVSRGWSLRQLDVQNAFLHGFLEEEVYMQQPPGFESSSKPDYVCKLDKALYGLKQAPRAWYSRLSKKLVELGFEASKADTSLFFLNKGGILMFVLVYVDDIIVASSTEKATTALLKDLNKEFALKDLGDLHYFLGIEVTKVSNGVILTQEKYANDLLKRVNMSNCKPVSTPLSVSEKLTLYEGSPLGPNDAIQYRSIVGALQYLTLTRPDIAYSVNKVCQFLHAPTTSHWIAVKRILRYLNQCTSLGLHIHKSASTLVHGYSDADWAGSIDDRKSTGGFAVFLGSNLVSWSARKQPTVSRSSTEAEYKAVANTTAELIWVQTLLKELGIESPKAAKIWCDNLGAKYLSANPVFHARTKHIEVDYHFVRERVSQKLLEIDFVPSGDQVADGFTKALSACLLENFKHNLNLARL

SEQ ID NO: 4 - DNA (genomic), 8416 bp, Oryza longistaminata; single-stranded, linear
  CDS join(4771..7384, 7676..8052): /product= “receptor kinase-like protein” /note= “Xa21 gene family member A1”
  misc_feature 7432..7614: /note= “Snap-Ol1, transposon-like element”
  original source strain: IRBB21
  chromosome/segment: 11
  map position: 11q, RG103
gccgtcgatctgtcattttgaaacggcccattcttttccatctatatgcattcatgaaatacatggtatatcccatcgatcggacatcacctgttagcgcgtacgccatcgtcgtcatcaacctagctagggcaaacgcaccttactgagctccgtcctccgatcgccaccatcaccaatgaacaagctgctgcggcctctcggtggcctgaggttgctcaaccgagaagaacatccgttccgatgcttctcctcctccatcgatctcgtcttcccaggtcgccgccgccgccacatggcaaccaccgtgacccacccgccgccaacggaatccgctggttcgacggcggcggccgcgactgctgacccgggctcggtgatgctggaacgttggggctgcctcaggggctccacggcggcgaacgtagtcgccgacgacaacaccgcgggagttccgcaccttccgcggccaacccctccggtcgcctcgtccgggttgcgccgggatctccttcatctgcttcgatcgcggggttgatggctacgtcatcgcggctcacggcgactctgtcctcttccggatgagttggaacgactacttcgtctacatggccgccgccggcaagccgccgtcgctgacgctgctccccgtctgcgacatccccatgaacgagcgctgctgggtcagcaaggaccgtttcaaggacgcttccgcaccacgggccgggtgttcgaccagcaggacaccggcatcctacgcctccgcggcgacgacggcggtgaggaggcgccgctctagtggcgcagtccagatcgcgcacgagccgccgttcgacacggccgagctctgcgtgctccgccccggccacggcgagtgggagctcaagatggcggtgcccatcgtccaccatgacagcttgaatcttataacaaaccttgctgaagctgacaaattctagcccccagccatgaagttggaaaaatcaatttccgattacacaaattggttaatacgcaaccatttagtgctcttaacatgaccaggttttacatgttcgttcggctcttagaatctgacaagaccttatctgctctgggcgtccccagccgaaattccattagttttctcggaggcttgtcagaacagcgtaaagggacaataggactgccttcaagatgaggcgatataagaggggatcaacagacaaatattgcacatataaaacttacagaagttgatgtagatgatgagacgaccaccacactaggcaaagaccaggtgtatagttgtactcaacaaatcacagaggtagtgagagatcgctacgatctactggtcgaagatcaggtgtaggcgtattctcgatcacctgaagaagaatctttaggtgttgagagatcgctactatctactggtcaatactagtaaaaaaacctcatagagatcggcactataggtgccggaacagctaaaaccggcacctataatacttttccctcctccgtggactcaaagcacgtaaaaccgacacctttaagcaactataggtgccggttctaaagaagaaccgacacctatagtataggtgctggttttttaaaaaaacccgacacctttaatataatataggtgtcggttcttctttaaaaccgacaccaataataaattatacgtgtcggttttttaataaaaccggcacctatccaaaccgagcctagctgtcgagtcgagccaatccaggctgacgcatatattagtctcgtccttatcgcctcgtctctctctctctctctctctctctcttctctctcgcctctctgtgtagcgcgcggcggtggtcgggcggcgtcccggcgtaggcaacagtggtggcgagatgggaggcggtggcgcatcacagctattcactttagcgcattgaataataatcggcatcatgatctacttatggttcgtgcaagggggaagattgtagatacacatggtcacagctaagtgctatgatcgcggctcatctctccaaatagattcatgccatccgtacttaaacagaaccttatgttccgtgcatcctttctgaagtcttgaaatgttttatcgatgttttaccactacgacccatctgtagggtgtctcgcatcccgtcttgtttatgtttttatgtgtgccagcgcaccattctagcacgcgctttgttcctgaacaaacaccttagccgtggtattaaatggaaatactgcatgatcatagcaggaactctcttcttcgaaggatgtccgtcaacatcacctaggtcatctcgtctaatcttatatcgtagtgccttacaaaccaggcgtgcttctaggttctcgtactcctcaacgcgatagaggatacaatcattcagacatacgtgtatcttctgtacttccagtccgagagggcagattacctttttagcttcgtacgttgtttcgagcaattcgtttccctcgggaagaatattctttacgagtttcaataactcgccaaatgccttgtcagtcacaccatattttgccttccattgtcagaattccagtgtggtacccaactttttgtgctcctattcgcaacctgggtacaaggacgttttgtggtatgctaacatccggtccaatttctagaccaactttttcacttttgcagtcattttttacatcgcacaacatttgaccaagattatccgtaccgccgttttcttcaacagctctgtccgcctcgcctattgtattttctgcaaatccatcatattgagcccagtctgaaatgttgtcgtcttcttattaattttcttctattgcaacccctaactctccgtgcaaggtctaacaattatagccaggtatgaatcccgattccaacaagtggatatgaagacttcttagatgtagaatactccttctggtttttgcacttttgcatggacaacatataaaacccttaggcttgtgggcattggccacactcaaaaaataatgcacgtcatcaataaactccttcgaccgtcattctgcagtgtacatccattaccgattcatctacattaggagataataattgtaaaggaagtccacaaaagtgaaactacttaaataatcatataataatttaaaatatcataattaaattgaaaactgacggttttaatgtattcttcttatttctaacgattttaacgagttttaaatggacttaatcggagtcatgattaactatttataaattttatctgtctcaataaattgtaaatatatttttccatgtattatccttgtttaattatttttaaaagttctaaacatatttttaatgcattctacttatttctaactattttaaagattttcaaatggacttagttttctatttttattcttcattttctatatttgccctttgttgtctctttttaacaattttatacaaatatttataattttattaagtaccctaattttccctaaacaatttttctctctcatcgtatttccatatatctttttgagataataatggatataaacatagctagaaatgtaaatgttcaccttgcatcaataggggatgaagttgctaaccttttagatctcctcgatttgtataatataaccaaaatattttcaccaaaaatttcgttaaacatccgagatatttgttgtttttgccgatcgagcaaagattagtagtccagcagtgtctgcaccaccaccatcgtgataatgcatcttgtgtgttattcttgatgagaaaatacgtagtgaaaaccacatatatggtggaaacttggaaactaccgttagatcgagaaatggatgtccaagatcgtccacgtcaccaagagataaaatttaactcgcagattcacttatgagttaaaattttaatgagagttaaattttaactcatgttgatgtggacgaatatcggacatccatttctcgatccaacgatagcttctaagtttccactacatatgtggtttgcactatatattttcccattcttgattatgtgtttgagagcagctagcacaaagagaaaaaaaagcatcgtttttcacgcgtatgttttcagaactgttaaatggtgtgtttttttaaaaaactttatatagaaaagtttctttaaaaaatatattaatctattttttaagtttaaaataattactacttaattaattatacactaacagcttatttcgttctacgtatcttgtcaattttcgctattcctttcttctcaaacacggcattggatgctctcatagcacttgctcgttcggatagaagacttgacgaagacgaccgctacaacttggtgtgttatatcgtgctttgtttagcataatcattacatatattccatgccgaagtgccgacgatgagaccgtgttcgatgcatctttgtatggcatctagggacaaagagcatagagtccctaccatagtacctgcttgcgcagaagacttgacgagaagaccgactgctacaccttggtgtgtaataatatcgtgttgtgtgtaccatgcatactcctttaaaacaaataatggtggtaacagtaaatctgtcatcccacccactctcattgtaaattttgcaagttatcacttgaacttcttaatactccatccgtttgcgtgtgttctttcagaatttgcgtgagcactttttcttctatataatctgtctagtccatgagctaaaccaacatctctcgctgtcttgccttgcacttctgcacgatgatatcactcccattattgctcttcgtcctgttgttctctgcgctgctgctctgcccttcaagcagtgacgacgatggtgatgctgccggcgacgaactcgcgctgctctctttcaagtcatccctgctataccaggggggccagtcgctggcatcttggaacacgtccggccacggccagcactgcacatgggtgggtgttgtgtgcggccgccggcacccgcacagggtggtgaagctgcggctgcgctcgtccaacctgaccgggatcatctcgccatcgctgggcaacctatccttcctcaggacgctgcaactcagcaacaaccacctgtccggcaagataccccaggagctcagccgtctcagcaggctccagcagctggtactgaatttcaacagcctatcgggtgagattccagctgctttgggcaatctaaccagtctctcagttcttgagctgactaacaatacactgtctggttctatcccttcatccctgggcaagctcaccggcctctataatcttgcactggctgaaaatatgctgtctggttccatccctacgtctttcggccaattgcgcagattatctttccttagcttagccttcaaccacttaagtggagcgatcccagatcctatttggaacatctcctctctcaccatatttgaagtcgtgtccaacaacctaactggtacactgcctgcaaatgcattcagtaatcttcctaatctgcagcaggttttcatgtactacaaccattttcatggtcctatccctgcatcgattggtaatgcttccagcatctcaatatttaccattggtttaaactcttttagcggtgttgttccaccggagattggaaggatgagaaatcttcagagactagagcttccagaaactcttttggaagctgaagaaacaaatgattggaaattcatgacggcattgacaaattgctccaatcttcaagaagtggaactggcaggttgcaaatttggtggagtcctccctgattctgtttccaatctttcctcttcgcttgtatctctctccattagagataacaaaatttcagggagcttacctagagatatcggtaatctcgttaatttacaatatctttctctcgctaacaactccttgacaggatcccttccctcttccttcagcaagcttaaaaatttacgtcgtctcactgtagataacaacaggttaattggttctctcccattgactatcggtaatcttacacaactaactaatatggaggtccaatttaatgcctttggtggtacaataccaagcacacttggaaacctgaccaagctgtttcaaataaatcttggccacaataactttatagggcaaattcccattgaaatatttagcattcccgcactctctgaaattttggatgtgtcccataataacttggagggatcaataccaaaagaaatagggaaacttaaaaatattgtcgaattccatgctgattcgaacaaattatcgggtgagatccctagcaccattggtgaatgccaacttctgcagcatcttttcctgcaaaacaatttcttaaatggtagcatcccaatagctctgactcagttgaaaggtctggacacacttgatctctcaggcaacaatttgtcaggtcagatacctatgtccttaggggacatgactctgctccactcgctgaacctttcgttcaacagcttccacggtgaagtgccaaccaatggtgtttttgcaaatgcttctgaaatttacatccaaggcaatgcccatatttgcggtggcatacctgaactacatcttccgacgtgttccttaaaatcaagaaagaaaaggaaacatcaaattctgctgttagtggttgttatctgtctcgtttcgacacttgccgtcttttcgttactctacatgcttctaacctgtcataagagaagaaagaaagaagtccctgcaacgacatccatgcaaggccacccaatgatcacttacaagcagctggtaaaagcaacggatggtttttcgtccagccatttgttgggttctggatcttttggctctgtttacaaaggagaatttgatagtcaagatggtgaaatcacaagtcttgttgccgtgaaggtactaaagctagaaactcctaaggcactcaagagtttcacggccgaatgcgaaacactacgaaatacgcgacaccggaatcttgtcaagatagttacgatttgctcgagcatcgataacagagggaatgatttcaaagcaattgtgtatgacttcatgcccaatggcagtctggaagattggctacaccctgaaacaaatgatcaagcagagcaaaggcacttgactctgcatcagagagtgaccatactacttgatgttgcatgtgcattggagcatcttcacttccatggccctgaacctattgtacactgtgatattaaatcaagcaatgtgttgttagatgctgatatggtagctcatgttggagactttggacttgcaagaatacttgttgagggaagctcattgatgcaacagtcaacaagttcgatgggaatcagggggacaattggttacgcagcaccaggttaatcctaaactgtttatgtctacctcctttcattgttttttttttagatttgctctggtccaacaaaaaatacctaaagatacagatacttgtacctcacagtactaaatagtttttgatcattgcattgttagatccaacgatcagaaaacgatttggtaccgtgaccgtgaggtatcggaatctcgagatatttttttgttcgaccgtagcaaatctatttttttgtttgttttcttctctttaatgttttatgactatgaaataatttttatttctggaaaacagagtatggtgtcgggaacactgcctcgacacatggagatatttacagttatggaattctagtgttggaaacagtaaccgggatgcggccggcagacagtacattcagaactggattgagcctccgtcagtacgttgaaccgggtctacatggtagactaatggatgttgttgacaggaagcttggtttggattccgagaaatggcttcaggctcgagatgtttcgccacgcagcagtattactgaatgccttgtttcactgcttagacttgggctgtcttgctctcaggaattgccatcgagtagaacgcaagccggagatgtcatcaatgaactgcgtgccatcaaagagtctctctcgatgtcatccgacatgtgaagatgtgagacatgctgatgttatgttggagtatttcgttgtaatgtaatgtgaagggtgagtgtgtgactgcttggttgtaagctatttcctgatctgcccatcagatcatgtatctgttctattgttgtatttctcagaacaaccacacacctaagtaggagtacacaatagtgtatttgtgtgatttcaatattggtgcatacccatgctatgtgaacagtcaatcggggagcgattcacaccataccgtgaaatcgacctaatcagctaatctaattctacaggctgcctttgcatgacagtgtgatattaaattagcccagccctttttagcaaacgatgggagggtcaatgctctaga

SEQ ID NO: 5 - protein, 996 aa
MISLPLLLFVLLFSALLLCPSSSDDDGDAAGDELALLSFKSSLLYQGGQSLASWNTSGHGQHCTWVGVVCGRRHPHRVVKLRLRSSNLTGIISPSLGNLSFLRTLQLSNNHLSGKIPQELSRLSRLQQLVLNFNSLSGEIPAALGNLTSLSVLELTNNTLSGSIPSSLGKLTGLYNLALAENMLSGSIPTSFGQLRRLSFLSLAFNHLSGAIPDPIWNISSLTIFEVVSNNLTGTLPANAFSNLPNLQQVFMYYNHFHGPIPASIGNASSISIFTIGLNSFSGVVPPEIGRMRNLQRLELPETLLEAEETNDWKFMTALTNCSNLQEVELAGCKFGGVLPDSVSNLSSSLVSLSIRDNKISGSLPRDIGNLVNLQYLSLANNSLTGSLPSSFSKLKNLRRLTVDNNRLIGSLPLTIGNLTQLTNMEVQFNAFGGTIPSTLGNLTKLFQINLGHNNFIGQIPIEIFSIPALSEILDVSHNNLEGSIPKEIGKLKNIVEFHADSNKLSGEIPSTIGECQLLQHLFLQNNFLNGSIPIALTQLKGLDTLDLSGNNLSGQIPMSLGDMTLLHSLNLSFNSFHGEVPTNGVFANASEIYIQGNAHICGGIPELHLPTCSLKSRKKRKHQILLLVVVICLVSTLAVFSLLYMLLTCHKRRKKEVPATTSMQGHPMITYKQLVKATDGFSSSHLLGSGSFGSVYKGEFDSQDGEITSLVAVKVLKLETPKALKSFTAECETLRNTRHRNLVKIVTICSSIDNRGNDFKAIVYDFMPNGSLEDWLHPETNDQAEQRHLTLHQRVTILLDVACALEHLHFHGPEPIVHCDIKSSNVLLDADMVAHVGDFGLARILVEGSSLMQQSTSSMGIRGTIGYAAPEYGVGNTASTHGDIYSYGILVLETVTGMRPADSTFRTGLSLRQYVEPGLHGRLMDVVDRKLGLDSEKWLQARDVSPRSSITECLVSLLRLGLSCSQELPSSRTQAGDVINELRAIKESLSMSSDM

SEQ ID NO: 6 - DNA (genomic), 19639 bp, Oryza longistaminata; single-stranded, linear
  - 5213..18201: /note= “Xa21 gene”
  CDS join(5213..7889, 8732..9132): /product= “receptor kinase-like protein” /note= “Xa21 disease resistance gene”
  misc_feature 9645..9769: /note= “Pop-Ol1, transposon-like element”
  misc_feature 13040..13248: /note= “Ds-rice1, transposon-like element”
  CDS join(15118..17720, 17827..18204): /product= “receptor kinase-like protein” /note= “Xa21 gene family member C; 2 bp deletion causing frame-shift mutation of ORF compared to family member A1”
  misc_feature 16183..16184: /note= “location of 2 bp deletion compared to family member A1”
  original source strain: IRBB21
  chromosome/segment: 11
  map position: 11q, RG103
aagctttgctcatttcttctccaattacaattaatcgtgtgtgcatcaagtaattaattaagaaactctggcttgttgaaaggtcgcagtgacaattaatcgtgagtgcataggatggggaaaacacgagggatcggtcgaccatcggggggagcaaaaatcaagcgctcccccgccccacgacgcgcacatgccacgccaccccaccacgcaccacgtgtgccgttgtaaacacctgccacgtgtccccatcacatactccgttgtaacaaatcacccatatattttggaaaccctatattaggagaattcgtttcattttttttctaccaaaaatatttcacctagtgtactcacaatgtttcactatgtatagatctaattttgtagtaaattgaaacattcttttgcaataaattacccatatattatggaaaccctctattaagggaattcgtttcattttttattccactaaaaatgtttcgcctagtgtacttgtaatgtttcactatgtatggattaaatgttgcagtgacttgaaacattctttcgctatttgctgaaacattgtttttatataaggtgaaacagcgcccgatttaaacgattgaaatattttcgatctacttagtgaaacaattccaatatacttggtgcaacaacgtgcaacatttaattataattccataataagcttgtcgacatttgatagtcacgactagggtatttggatggtatggggatcatcggtaccagggtatatgcgagattgaggtaaaagagatggagatagggatttttatataggttcgggccccttatctgataggtaatagccctacatcctgtttatatgtgattgatatagaaaaaccacagaatacaacaattgggataacctatctagccgttgttgacttggcggcacgaacaccaactcgtagtcgacgacggggaagcctttctcctcgattgtgaactcgacaagattagagatatcgctagatccctcttgccggcctctgtaggtaccggatggggtgtgtctaggctaatctcagatgtcgatgtttggcggcgtattggcttgtgtcttgtggcttctatgttgtgtgtcccctctcctccaatggaggcttggatttagactcatagatttccccttgtccaagtagaactagggagaccaatatagatacaatccgagtagtacttgtcgtttctatatagaactctattttgtccttccttatccggaactccttctatatatgaggtatgtttccgtataagacttggtatgtggtgggcctcgccgagcttagtcgattactattgggtatgtggtatcctaggccccagctgccattttccacaaagacagcttgaatcttataacaaaccttgctgaagctgacaaatcctagcccccagccatgaagttggaaaaatcaatttccgattacacaaattggttaatacgcaaccatttagtgctcttaacatgaccaggttttacatgttcattcggctcttagaatctgacaagaccttatctgctctgggcgtccccagccgaaattccattagttttctcggaggcttgtcagaacagcgtaaagggacaataggactgccttcaagatgaggcgatataagaggggatcaacagacaaatattgcacatataaaacttacagaagttgatgtagatgatgagacgaccaccacactaggcaaagaccaggtgtatagttgtactcaacaaatcgcagaggtagtgagagatcgctacgatctactggtcgaagatcaggtgtaggcgtattctcgatcacctgaagaagaatctttaggtgttgagagatcgctactatctactggtcaacactagtaaaaaaacctcatagagatcggcactataggtgccgaacagctaaaacccgcacctatatactttcctctcgtggactcaaagcacgtaaaccgacactttagcaactataggtgcggtctaaagagaccgacactatagtatagtgctggttttaaaaaacccgacactttaatataatatagtgtcggtctctttaaaacgacacaatataaatatacgtgtcggtttttaataaacggcacctatcaaaccgagcctagctgtcgagtcgagccaatccaggtgacgcatatattagtctcgtccttatcgcctccggtctctctctctctctctctctctctttctctctcgcctctctgtgtagcgcgcggcggtggtcgggcagcgtcccggcgtaggcaacagtggtggcgagatgggaggcggtggcgcatcacagctattcactttagcgccttgaataataatcggcatcatgatctacttatgcttcgtgcaagagggaagaatgtagatacacatggtcacagctaagtgctatgatcgcggctcatctctccaaatagattcatgccatccgtacttaaacagaaccttatgttccgtgcatcctttctgaagtcttgaaatgttttatcgatgttttaccactacgacccatctgtagggtgtctcgcatcccgtcttgtttatgtttttatgtgtgccagcgcaccattctagcacgcgctttgttcctgaacaaacaccttagccgtggtattaaatggaaatactgcatgatcatagcaggaactctcttcttcgaaggatgtccgtcaacatcacctaggtcatctcgtctaatcttatatcgtagtgccttacaaaccaggcgtgcttctaggttctcgtactcctccacgcgatagaggatacaatcattcagacatacgtgtatcttctgtacttccagtccgagagggcagattacctttttagcttcgtacgttgtttcgagcaattcgtttccctcgggaagaatattctttacgagtttcaataactcgccaaatgccttgtcagtcacaccatattttgccttccattgtcagaattccagtgtggtacccaactttttgtgctcctattcgcaacctgggtacaaggacgttttgtggtatgctaacatccggtccaatttctagaccaactttttcacttttgcagtcattttttacatcgcacaacatttgaccaagattatccgtaccgccgttttcttcaacagctctgtccgcctcgcctattgtattttctgcaaatccatcatattgagcccagtctgaaatgttgtcgtcttcttattaattttcttctattgcaacccctaactctccgtgcaaggtctaacaattatagccaggtatgaatcccgattccaacaagtggatatgaagacttcttagatgtagaatactccttctggtttttgcacttttgcatggacaacatataaaacccttaggcttgtgggcattggccacactcaaaaaataatgcacgtcatcaataaactccttcgaccgtcgttctgcagtgtacatccattaccgattcatctacattaggagataataattgtaaaggaagtccacaaaagtgaaactacttaaataatcatataataatttaaaatatcataattaaattgaaaactgacggttttaatgtattcttcttatttctaacgattttaacgagttttaaatggacttaatcggagtcatgattaactatttataaattttatctgtctcaataaattgtaaatatatttttccatgtattatccttgtttaattatttttaaaagttctaaacatatttttaatgcattctacttatttctaactattttaaagattttcaaatggacttagttttctatttttattcttcattttctatatttgccctttgttgtctctttttaacaattttatacaaatatttataattttattaagtaccctaattttccctaaacaatttttctctctcatcgtatttccatatatctttttgagataataatggatataaacatagctagaaatgtaaatgttcaccttgcatcaataggggatgaagttgctaaccttttagatctcctcgatttgtataatataaccaaaatattttcaccaaaaatttcgttaaacatccgagatatttgttgtttttgccgatcgagcaaagattagtagtccagcagtgtctgcaccaccaccatcgtgataatgcatcttgtgtgttattcttgatgagaaaatacgtagtgaaaaccacatatgtggtggaaacttagaaactaccgttagatcgagaaatggatgtccaagattcgtccacgtcaccaagagataaaatttagctcgcagattcacttatgagttaaaattttaatgagagttaaattttaactcatgttgatgtggacgaatatcggacatccatttctcgatccaacgatagcttccaagtttccactacatatgtggtttgcactatatattttcccattcttgattatgtgtttgagagcagctagcacaaagagaaaaaaaagcatcgtttttcacgcgtatgttttcagaactgttaaatggtgtgttttttgaaaaaactttctatagaaaagtttctttaaaaaatatattaatctattttttaagtttaaaataattactacttaattaattatacactaacagcttatttcgttctacgtatcttgtcaattttcgctattcctttcttctcaaacacggcattggatgctctcatagcacttgctcgttcggatagaagacttgacgaagacgaccgctacaacttggtgtgttatatcgtgctttgtttagcataatcattacatatattccatgccgaagtgccgacgatgagaccgtgttcgatgcatctttgtatggcatctagggacaaagagcatagagtccctaccatagtaccagctcgcgcagaagacttgacgagaagaccgactgctacaccttggtgtgtaataatatcgtgttgtgtgtaccatgcatactcctttaaaacaaataatggtggtaacagtaaatctgtcatcccacccactctcattgtaaattttgcaagttctcacttgaacttcttaatactccatccgtttgcgtgtgttctttcagaatttgcgtgagcactttttcttctatataatctgtctagtccatgagctaaaccaacatctctcgctgtcttgccttgcacttctgcacgatgatatcactcccattattgctcttcgtcctgttgttctctgcgctgctgctctgcccttcaagcagtgacgacgatggtgatgctgccggcgacgaactcgcgctgctctctttcaagtcatccctgctataccaggggggccagtcgctggcatcttggaacacgtccggccacggccagcactgcacatgggtgggtgttgtgtgcggccgccgccgccgccggcacccacacagggtggtgaagctgctgctgcgctcctccaacctgtccgggatcatctcgccgtcgctcggcaacctgtccttcctcagggagctggacctcggcgacaactacctctccggcgagataccaccggagctcagccgtctcagcaggcttcagctgctggagctgagcgataactccatccaagggagcatccccgcggccattggagcatgcaccaagttgacatcgctagacctcagccacaaccaactgcgaggtatgatcccacgtgagattggtgccagcttgaaacatctctcgaatttgtacctttacaaaaatggtttgtcaggagagattccatccgctttgggcaatctcactagcctccaggagtttgatttgagcttcaacagattatcaggagctataccttcatcactggggcagctcagcagtctattgactatgaatttgggacagaacaatctaagtgggatgatccccaattctatctggaacctttcgtctctaagagcgtttagtgtcagagaaaacaagctaggtggtatgatccctacaaatgcattcaaaacccttcacctcctcgaggtgatagatatgggcactaaccgtttccatggcaaaatccctgcctcagttgctaatgcttctcatttgacagtgattcagatttatggcaacttgttcagtggaattatcacctcggggtttggaaggttaagaaatctcacagaactgtatctctggagaaatttgtttcaaactagagaacaagatgattgggggttcatttctgacctaacaaattgctccaaattacaaacattgaacttgggagaaaataacctggggggagttcttcctaattcgttttccaatctttccacttcgcttagttttcttgcacttgaattgaataagatcacaggaagcattccgaaggatattggcaatcttattggcttacaacatctctatctctgcaacaacaatttcagagggtctcttccatcatcgttgggcaggcttaaaaacttaggcattctactcgcctacgaaaacaacttgagcggttcgatcccgttggccataggaaatcttactgaacttaatatcttactgctcggcaccaacaaattcagtggttggataccatacacactctcaaacctcacaaacttgttgtcattaggcctttcaactaataaccttagtggtccaatacccagtgaattattcaatattcaaacactatcaataatgatcaatgtatcaaaaaataacttggagggatcaataccacaagaaatagggcatctcaaaaatctagtagaatttcatgcagaatcgaatagattatcaggtaaaatccctaacacgcttggtgattgccagctcttacggtatctttatctgcaaaataatttgttatctggtagcatcccatcagccttgggtcagctgaaaggtctcgaaactcttgatctctcaagcaacaatttgtcaggccagatacccacatccttagcagatattactatgcttcattccttgaacctttctttcaacagctttgtgggggaagtgccaaccattggtgctttcgcagctgcatccgggatctcaatccaaggcaatgccaaactctgtggtggaatacctgatctacatctgcctcgatgttgtccattactagagaacagaaaacatttcccagttctacctatttctgtttctctggccgcagcactggccatcctctcatcactctacttgcttataacctggcacaagagaactaaaaagggagccccttcaagaacttccatgaaaggccacccattggtctcttattcgcagttggtaaaagcaacagatggtttcgcgccgaccaatttgttgggttctggatcatttggctcagtatacaaaggaaagcttaatatccaagatcatgttgcagtgaaggtactaaagcttgaaaatcctaaggcgctcaagagtttcactgccgaatgtgaagcactacgaaatatgcgacatcgaaatcttgtcaagatagttacaatttgctcgagcattgataacagagggaacgatttcaaagcaattgtgtatgacttcatgcccaacggcagtctggaagattggatacaccctgaaacaaatgatcaagcagaccagaggcacttgaatctgcatcgaagagtgaccatactacttgatgttgcctgcgcactggactatcttcaccgccatggccctgaacctgttgtacactgtgatattaaatcaagcaatgtgctgttagattctgatatggtagcccatgttggagattttgggcttgcaagaatacttgttgatgggacctcattgatacaacagtcaacaagctcgatgggatttatagggacaattggctatgcagcaccaggtcagcaagtccttccagtattttgcattttctgatctctagtgctatatgaaatagtttttacctctagtgaaactgatggagaatataagtaattaattgaactaattaaattgcacaaaaataagattatttgccatatctattcagatgctaaatatagctagttcatagaggtacagatttttttatataggactctagagctaccacacactcaaatcaaattatgggtgttttctgctctacactgcaatatgaaatgattattacttctacatgaactgatggaggagtttcagaaggatcaaatttgagtaaatttttcaattctacatttaagaaacacttttttttcatatgctagttacatttttttatttcacgagcttacattgaccatgaaaaatacttggcactacttactaattcccacatggaggtagtgaaaataatatagatacaaaaacgaaatatcctatgttgtgtgatatactataatcacaatgaacacaaacaggattcgtacaaaagtaattagccatcatagcaactgattgcttggggtaactgtatagcacaatcataccaaatttctttagatatgtatctgtaaattagattcttaaagttaaatatgaaatttcattggtatttatgtttctttatataataaaaattaatccagcctttgcatctatcatttgtccagacatccttgttatttgtgatatttaacacgtaaatttacataattatacatccaagttctttttatttaacactgtaaatttcaaatcgtacatgttataaagaatgtactatatttcctgctcaaacagagtatggcgttgggctcattgcatcaacgcatggagatatttacagctatggaattctagtgctggaaatagtaaccgggaagcggccaactgacagtacattcagacccgatttgggcctccgtcagtacgttgaactgggcctacatggcagagtgacggatgttgttgacacgaagctcattttggattctgagaactggctgaacagtacaaataattctccatgtagaagaatcactgaatgcattgtttggctgcttagacttgggttgtcttgctctcaggaattgccatcgagtagaacgccaaccggagatatcatcgacgaactgaatgccatcaaacagaatctctccggattgtttccagtgtgtgaaggtgggagccttgaattctgatgttatgtctcgtaatgttttattgccacacttcagatcgacttctgcagtggtatctaccacacgatcactaaagtcaccgtggctatttcctgatccagcatatctgatcatgcatgttctgtgttgtatacctgtattttactctgaattgccacaccgcaaccctgcctctgtttgtttggtatacaaaagatagtgatgagtttattgttttaggggcttcctagttggcgcgtgtgcatgccggcatgcacgcagcccgagggtgggtttcttttttttccattgttattccgttgctttttttcaccacggtagatttttttttccggatttccattttttccgttgtttttctctatcgcttatgttggcggatttttttccgtggttttctttccgaagacgagtatatctaacgtaactaacatgttacttttagataacgatggttattaagataagatttttctctggaagatttttgtaagtaacagattgaaaacaaatctatacgtgaggtcaaattttgaaaactttcaatctagatttaaaagcttttcaactcaaaatttgaatttttgaagtgaaaatttgaatactttcaaaaattactagtaatcgacaaaaaaaatatggaaatggaaacggaaatagttttgctgttataccgatcgtttccatatttaccgtattcttatagaaattaccgtttcttataatatggtaattaccgtatttctaaatatgttgatatttatagggcatgtctctacttgactcacagtttagagattgattgactatttaatcaaatccctaacttgattgcatggctaaaatggagttgatttctaatttatatagtatagcttgaatttatttgtaaatataacatacttatgtaaagttaaatatatgttttctatagtttaatgtttctgtatttgttaccggttttcgatctgtaccgacatatttccatcagtattattccatttccggttttccgatatttccgatatcgttttcgtttccgactttaccgttttcgatttcatttccgagaaaaatatgattatggaaatggtcgaggctgttttccgatcgtttccgaccgttttcatccctacccgtagtaataatatataacattttatctctaatctttctctctctcatatcaatgaataatcgctaagagactgctattaacaaggcttatatatatatatgccgtcgatcagtcattttgaaacggcccacttcttttccatctatatgcattcatgaaatacatggtatatcccatcgatcggacatcacctgttagcgcgtacgccatcgtcgtcatcaacctagctagggcaaacgcaccttgctgagctccgatcctccgatcgccaccatcaccaatgaacaagctgctgcggcctctcggtggcctgaggttgctcaaccgagaagaacatccgttccgatgcttctcctcctccatcgatctcgtcttcccaggtcgccgccgccgccacatggcaaccaccgtgacccacccgccgccgacggaatccgctggttcgacggcggcggccgcgactgctgacccggcctcggtgatgctggaacattggggctgcctcaggggctccacgccggcgaacgtagtcgccgacgacaacaccgccgcggagtcccgcacctcccgcggccaacccctccgggtcgccctcgcccgcgcgtcgccgccggcgatctccttcatctgcttcgatcgcggggatgatggctacgtcatcgcggctcacggcgactctgtcctcttccggatgagttggaacgactacttcgtctacatggccgccggcggccgccgtcgctgacgctgctccccgtctgcgacatccccatgaacgagcgctgctgggtcagcaaggaccgtttcaaggacagcttccacaccacgggccgggagttcgaccagcaggacaccggcatcctgcgcctccgcggcgacgacggcggcgaggaggcgccgctctagtggcgcagtccagatcgcgcacgagccgccgttcgacacggccgagctctgcgtgctccgccccggccacggcgagtgggagctcaagatggcggtgcccatcgtccaccatgacagcttgaatcttataacaaaccttgctgaagctgacaaatcctagcccccagccatgaagttggaaaaatcaatttccgattacacaaattggttaatacgcaaccatttagtgctcttaacatgaccaggttttacatgttcgttcggctcttagaatctgacaataccttatctgctctgggcgtccccagccgaaattccattagttttctcggaggcttgtcagaacagcgtaaagggacaataggactgccttcaagatgaggcgatataagacgggatcaacagacaaatattgcacatataaatacttacagaagttgatgtagatgatgagacgaccaccacactaggcaaagaccaggtgtatagttgtactcaacaaatcgcagaggtagtgagagatcgctacgatctactggtcaaagatcaggtgtaggcgtattctcgatcacctgaagaagaatctctaggtgttgagagatcgctactatctactggtcaaaactagtaaaaaaacctcatagagatcggcactataggtgccgaacagctaaaacccgcacctatatactttcctctcgtggactcaaagcacgtaaaccgacactttagcaactataggtgcggtctaaagagaccgacactatagtatagtgctggttttaaaaaacctgacactttaatataatatagtgtcggtctctttaaaacgacacaatataaatatacgtgtcggttttaataaacggcacctatcaaacgatcctagctgtcgagtcgagccaatccaggtgacgcatatattagtctcgtccttatcgcctcatctctctctctctctctctctctctctttctctctcgcctctctgtgtagcgcgcggcggtggtcgggcggcatcccggcgtaggcaacagtggtggcgagatgggaggcggtggcgcatcacagctattcactttagcgccttgaataataatcggcatcatgatctacttatgcttcgtgcaagggggaagattgtagatacacatggtcacagctaagtgctatgatcgcggctcatctctccaaatagattcatgccatccgtacttaaacagaaccttatgttccgtgcatcctttctgaagtcttgaaatgttttatcgatgttttaccactacgacccatctgtagggtgtctcgcatcccgtcttgtttatgtttttatgtgtgccagcgcaccattctagcacgcgctttgttcctgaacaaacaccttagccgtggtattaaatggaaatactgcatgatcatagcaggaactctcttcttcgaaggatgtccgtcaacatcacctaggtcatctcgtctaatcttatatcgtagtgccttacaaaccaggcgtgcttctaggttctcgtactcctcaacgcgatagaggatacaatcattcagacatacgtgtatcttctgtacttccagtctgagagggcagattaccattttagcttcgtacgttgtttcgagcaattcgtttccctcgggaagaatattctttacgagtttcaataactcgccaaatgccttgtcagtcacaccatattttgccttccattgtcagaattccagtgtggtacccaactttttgtgctcctattcgcaacctgggtacaaggacgttttgtggtatgctaacatccggtccaatttctagaccaactttttcacttttgcagtcattttttacatcgcacaacatttgaccaagattatccgtaccgccgttttcttcaacagctctgtccgcctcgcctattgtattttctgcaaatccatcatattgagcccagtctgaaatgttgtcgtcttcttattcattttcttctattgcaacccctaactctccgtgcaaggtctaacaattatagccaggtatgaatcccgattccaacaagtggatatgaagacttcttagatgtagaatactccttctggtttttgcacttttgcatggacaacatataaaacccttaggcttgtgggcattggccacactcaaaaaataatgcacgtcatcaataaactccttcgaccgtcgttctgcagtgtacatccattgccgattcatctacattaggagataataattgtaaaggaagtccacaaaagtgaaactacttaaataatcatataataatttaaaatatcataattaaattgaaaactgacggttttaatgtattcttcttatttctaacgattttaacgagttttaaatggacttaatcggagtcatgattaactatttataaattttatctgtctcaataaattgtaaatatatttctccatgtattatccttgtttagttatttttaaaagttctaaacatatttttaatgcattatacttatttctaactattttaaagattttcaaatggacttagttttctatttttattcttcattttgtatatttgccctttgttgtctctttttaacaattttatacaaatatttataattttattaagtaccctcattttccctaaacaatttttctctctcatcgtatttccatatatctttttgagataataatggatataaacatagctagaaatgtaaatgttcaccttgcatcaataggggatgaagttgctaaccttttagatctcctcgatttgtataatataaccaaaatattttcaccaaaaatttcgttaaacatccgagatatttgttgtttttgccgatcgagcaaagattagtagtccagcagtgtctgcaccaccaccatcgtgataatgcatcttgtgtgttattcttgatgagaaaatacgtagtgaaaaccacatatgtggtggaaacttggaaactaccgttagatcgagaaatggatgtccaagattcgtccacatcaccaagagataaaatttaactcgcagattcacttatgagttaaaattttaatgagagttaaattttaactcatgttgatgtggacgaatatcggacatccatttctcgatccaacgatagcttccaagtttccactacatatgtggtttgcactatatattttcccattcttgattatgtgtttgagagcagctagcacaaagagaaaaaaaagcatcgtttttcacgcgtatgttttcagaactgttagatggtgtgttttttgaaaaaactttctatagaaaagtttctttaaaaaatatattaatctattttttaagtttaaaataattactacttaattaattatacactaacagcttatttcgttctacgtatcttgtcaattttcgctcatcctttcttctcaaacacggcattggatgctctcatagcacttgctcgttcggatagaagacttgacgaagacgaccgctacaacttggtgtgttatatcgtgctttgtttagcataatcattacatatattccatgccgaagtgccgacgaggagaccgtgttcgatgcatctttgtatggcatctagggacaaagagcatagagtccctaccatagtacctgctcgcgcagaagacttgacgagaagaccgactgctacaccttggtgtgtaataatatcgtgttgtgtgtaccatgcatactcctttaaaacaaataatggtggtaacagtaaatctgtcatcccacccactctcattgtaaattttgcaagttatcacttgaacttcttaatactccatccgtttgcgtgtgttctttcagaatttgcgtgagcactttttcttctatataatctgtctagtccatgagctaaaccaacatctctcgctgtcttgccttgcacttctgcacgatggtatcactcccattattgctcttcgtcctgttgttctctgcgctgctgctctgcccttcaagcagtgacgacgatggtgatgctgccggcggcgaactcgcgctgctctctttcaagtcatccctgctataccaggggggccagtcgctggcatcttggaacacgtccggccacagccaacactgcacatgggtgggtgttgtgtgcggccgccggcacccgcacagggtggtgaagctgcggctgcgctcgtccaacctgaccgggatcatctcgccgtcgctgggcaacctatccttcctcaggacgctgcaactcagcaacaaccacctgtccggcaagataccccaggagctcagccgtctcagcaggctccagcaactggtactgaatttcaacagcctatcgggtgagattccagctgctttgggcaatctaaccagtctctcggttcttgtgctgactaacaatacactgtctggttctatcccttcatccctgggcaagctcaccggcctctataatcttgcactggctgaaaatatgctgtctggttccatcccttcatctttcggccaattgcgcagattatctttccttagcttagccttcaaccacttaagtggagcaatcccagatcctatttggaacatctcctctctcaccatatttgaggtcatatccaacaagctaaatggtacactgcctacaaatgcattcagtaatcttcctagtctgaaggaggtatacatgtattacaaccagtttcatggtcatatcccggcatcgataggtaatgcttccaacatctcaatatttaccattggtttaaactcctttagcggtgttgttccactggagattggaaggctgagaaatcttcagaggctagagcttggagaaactcttctagaatctaaagaaccaaacgattggaaattcatgatggcattgacgaattgctccaatcttcaagaagtagaattgggactttgtaaatttggtggagtcattcctgattctgtttccaatctttcctcttccctattatatctcttttttcgataacataatttcagggagcttacctaaggatatcggtaatctcgttaatttagaaactctttctctcgctaacaactccttgacaggatcccttccctcatccttcagcaagcttaaaaatttacatcgtctcaaactttttaacaacaaaataagtggttctctcccattaaccattggtaatcttacacaactaactaatatggagctccactttaatgccttcggtggtacaataccaggcacacttggaaacctgaccaagttgtttcaaataaatcttggccataataactttataggtcaaattcccattgaaatatttagcattcctgcactctctgaaattttggatgtgtctcataataacttggagggatcaataccaaaagaaatagggaaacttaaaaatattgtcgaattccatgctgattcgaacaaattatcgggtgagatccctagcaccattggtgaatgccaacttctgcagcatcttttcctgcaaaacaatttcttaaatggtagcatcccaatagctctgactcagttgaaaggtctggacacacttgatctctcaggtaagaatttgtcaggtcagatacctatgtccttaggggacatgcctctgctccactcgctgaaccattcgttcaacagcttccacggtgaagtgccaaccaatggtgtttttgcaaatgcttctgaaatttacatccaaggcaatgcccatatttgcggtggcatacctgaactacatcttccgacgtgttccttaaaatcaagaaagaaaaagaaacatcaaattctgctgttagtggttgttatctgtctcgtttggacacttgccgtcttttcgttactctacatgcttctaaccggccataagagaagaaagaaagaagtccctacaacgacatccatgcgaggccacccaatgatcacttacaagcagctggtaaaagcagcagatggttgttcgtccagccatttgctgggctctggatcctttggctctgttttcaaaggagaatttgatagccaagattgtgaaagcacaagtcttgttgccgtgaaggtactaaagctggaaactcctaaggcactcaagagtttcatggccgaatgcgaaacactgcgaaatactcgacacgtcaagatagttacaatttgctcgagcatcgataacagagggaatgatttcaaagcaattgtgtatgacttcatgcccaatggcagtctggaagattggctacaccctgaaacaaatgatcaagcagagcaaaggcacttgactctgcatcagagagtgaccatactgcttgatgttgcatgtgcattggaccatcttcacttccatggccctgaacctattgtacactgtgatattaaatcaagcaatgtgttgttagatgctgatatggtagcccatgttggagactttggactcgcaagaatacttattgagggaagctcattgatgcaacagtcaacaagttcgatggtaatcagggggacaattggttacgcagcaccaggttaagcctaaactgtttatgtctacctcatttcatttcttcttttttgtgttttcttctctctagtgttttatgactatgaaataatttttgctactggaaaacagagtatggtgtcgggaacactgcctcgacacatggagatatttacagttatggaattctagtgttggaaacagtaaccgggaagcggccgacagatagtacattcagaactggattgagcctccgtcagtacgttgaaccgggtctacatggtagactaatggatgttgttgacaggaagcttggtttggattccgagaaatggcttcaggctcgagatatttcgccatgcagcagtattagtgaatgccttgtttcactgcttagacttgggttgtcttgctctcaggaattgccatcgagtagaatgcaagccggagatgtcatcaatgaactgcgtgccatcaaagagtccctctcgatgtcatccggcatgtgaagatgttggagtatttcgttgtaatgtgatgtgtctattagtacccttcacaactgatttcattctgccgtggtatttagttatttacaagagagtcactgaagggtgagtgtgtgactgcttggctgtagctatttcctgatctgcccatcagatcatgtatctgttctattgttgtatttctcagaataaccacacacctaagtacacaacactgtatttgtgtgatttcaatattgatgcatatatacccatgctatatgctagaattatatacaaaaattttgagatgtctgaagttaacaatcaatcaggaagcgattcacaccaaaccgcgaaatcgacctaatcagctaatctaattgtacatgctgcctttgcatgacagtgcgatattaaattagcccagccctttttagcaaaggattggagggttaatgttctagagaaaaggatgcttgttaggttctctcttctctctcggttctcttgttagattatggaaccaattgatttcctctcgaaccaatcgatttcgccaccgtcgccaccaggttccaaatcgatattccggcgagatgccgttacagcgttttatagacgcaactcacgcctagactttcttctcggtacagaacggccaagcccaagacattccacggcccatttaggccccttgtatttagtgttgcttttactaatgcgcttctcgcttgccttggcataatctggaactccgccttcgatctgtagcccgtcttcttccttgtcgtccttgaggttctcagtcgcagcacccagcaccctaaacagatgcctttggttaggaaacatgggtggcagcaacctttccatgttggaatatctcgaaatctcctacacagatttattttgctaggtgaaataccttctcaccttggtaattaacatttcaaatttgtgccaccttaatctcagtggcacatatatgtacacaatagatatctcttggttagctcatcaacatttgcttgaatatctcgacatgacttttataaatctaagggtacgttcagtcatctccgacacgcaaaacgaagcaccattcgcgcatgattaatcaaatattagctaaaaaattataaaatggattaatatattttttaaaagccacgctcctataatattttttaaaaaatacatagtttaacagtttgaaaagcgtgtcgtacggaaaacgggagaggtgaagttggcaaagtagactttagaacacagcctaagtatggcagttcattggcctcaagttttcaacatgattccatctttggaggccctccttgtttcctgcagctcacttccaggctcagcccagccgctgaacacaactaaacttcacaaaacttgtagtgcttcatgtttcaaggaatgatgactttgtcaattcaatggtacc

SEQ ID NO: 7 - protein, 1025 aa
MISLPLLLFVLLFSALLLCPSSSDDDGDAAGDELALLSFKSSLLYQGGQSLASWNTSGHGQHCTWVGVVCGRRRRRHPHRVVKLLLRSSNLSGIISPSLGNLSFLRELDLGDNYLSGEIPPELSRLSRLQLLELSDNSIQGSIPAAIGACTKLTSLDLSHNQLRGMIPREIGASLKHLSNLYLYKNGLSGEIPSALGNLTSLQEFDLSFNRLSGAIPSSLGQLSSLLTMNLGQNNLSGMIPNSIWNLSSLRAFSVRENKLGGMIPTNAFKTLHLLEVIDMGTNRFHGKIPASVANASHLTVIQIYGNLFSGIITSGFGRLRNLTELYLWRNLFQTREQDDWGFISDLTNCSKLQTLNLGENNLGGVLPNSFSNLSTSLSFLALELNKITGSIPKDIGNLIGLQHLYLCNNNFRGSLPSSLGRLKNLGILLAYENNLSGSIPLAIGNLTELNILLLGTNKFSGWIPYTLSNLTNLLSLGLSTNNLSGPIPSELFNIQTLSIMINVSKNNLEGSIPQEIGHLKNLVEFHAESNRLSGKIPNTLGDCQLLRYLYLQNNLLSGSIPSALGQLKGLETLDLSSNNLSGQIPTSLADITMLHSLNLSFNSFVGEVPTIGAFAAASGISIQGNAKLCGGIPDLHLPRCCPLLENRKHFPVLPISVSLAAALAILSSLYLLITWHKRTKKGAPSRTSMKGHPLVSYSQLVKATDGFAPTNLLGSGSFGSVYKGKLNIQDHVAVKVLKLENPKALKSFTAECEALRNMRHRNLVKIVTICSSIDNRGNDFKAIVYDFMPNGSLEDWIHPETNDQADQRHLNLHRRVTILLDVACALDYLHRHGPEPVVHCDIKSSNVLLDSDMVAHVGDFGLARILVDGTSLIQQSTSSMGFIGTIGYAAPEYGVGLIASTHGDIYSYGILVLEIVTGKRPTDSTFRPDLGLRQYVELGLHGRVTDVVDTKLILDSENWLNSTNNSPCRRITECIVWLLRLGLSCSQELPSSRTPTGDIIDELNAIKQNLSGLFPVCEGGSLEF

SEQ ID NO: 8 - DNA (genomic), 9424 bp, Oryza longistaminata; single-stranded, linear
  CDS 2819..5260: /product= “receptor kinase-like protein” /note= “Xa21 gene family member E”
  misc_feature 5211..8128: /note= “truncator, an insertion sequence with the characteristics of a transposon”
  misc_feature 5484..5665: /note= “Snap-Ol2, transposon-like element”
  intron 8357..8644
  original source strain: IRBB21
  chromosome/segment: 11
  map position: 11q, RG103
aagcttcacttttcccctattttgttaagatcagttaatggtgtcaattcagccataaaaagacatgtatcccttttacatttgcccacaaaaaaatttgctgggtcacattagatatacggacacacatttgaagtattaaacgtagactaatacaaagcaaattatataatccgcatgtaaactgcgagacgaatttattaagcctaattaatccgtcattagcaaatgtttactgtagcactacattgttagatcatggtgcaattaggcttaaaagatttgtctcgtaatttacacacaaactgtgtaattagttttttcaatatttaatacttcgtacatgtatttaaacgtttgatgtgacgggacaaaaaaaaaattgccggtgggtctaaatccaccaccccccctcctcttagagttgatatacattttcacacaacttgcacggctgcaaaacttgactaaaaagattcatgacaaatttctcgcaaatattgagataggaaaagagagagagaaaatcaggtagcaacggcatctcgctggtctttgtgaacgggaaactattttaacagctcgagtggacgtcaacccgtttattgcatgtcatctaaatggttataaaaaaaattgaaaaaatatgaataaggatagatcaatatgtaatatatcaatccacaaacatgcaagttaaaatttaacttctacaagttgtaataaaaataagaaacaaaactcaaattactatatgtatatttacaattaaatttgttatttttgttataacctgtagaagttaaatttgaacttgcatgtttgtggagtgatatattacatattgatctatcttgtcaatttttttgaaaatttttcgtaaccatctagttgacatgcaataaacggatggacgtccactcgagtgctgctagggcggatctatagtacgttgtcaggcgacaccgatgactcccggtaaaaacctatagcaaaactgcttattcatatgttataacaccatgatctaaacataattagtatactatgataccgttagacacgttatgacaccaatgaatcgattttctggatccgccacagtgcacgggattgggatgggaacggccgatggacgctgtgtgttgggctagctaggtggggttgggagttgggactaggtaggttatttttgccaggggtacaatagttcttttgcattttgtaactcttctttttctttcgagataatccattatatgccattaactttgtcgcacgtctacgatttgccactgactttgtcacgttctacaatatgccatcgactttttcttaacttctacgatttaccatcgccgtccggttagccacttttagtactgtacaaatttgttgaaatgaccaaaatacccctatgacaaaaatatccaaaatttggataaaattatcaaaatattatattataaacataagattgtaaacatccaaaatttgaccaaaaacttgaaatatgatatttcataatttttatccaaattttgaaaccttttctcccaggggtattttggtcattttgacaaatttgtacagtactaacggagactaaccggacggcgatggtaaattgtagaagttaagcaaaagtcgatggcatatcgtagaacatgataaagtcagtggcaaatcatagacgtgtgacaaagtcagtggtatataatggtttctctcttttctttttctctcagaaatttgggcccagctaatttttgccaatgttgttgcaagaactcaacaagtgtatcaacctctcttctgtattccatctgccttctatgggtgattatttacacatttatatacagggatatacaagaggaaaatgcttcggtgcccctatccttacgatgtgcctatgtgactggccggtgggccagccttggtcctaatggtaacttgatggaggacaggagagctgcccccggcatgggggcatcgctgttcccgagggaccttgctactacccgggtaaaggcatccccgggtaatagcatgctccgcctagctgccatcaatagtgtcagtgttagttggggcgcgtagtgcgcatttattcgcgcccctagcgcctggttcttgacttgcttgcgcgagcacatcgaggcataattcactccggttggcacatcgggctttaggggcccctcggagctccgaagcccttaggtagcctccggagctccccttccggactccgggtacctttcaggcttcggagctcggtcactaggtgtgtcaccggagcccatggcgctcgacctcccggagctcttccggagccggtcggccgccatccgaagcctgcttaggtcttgttcctctcgccctcaatgagatccgattctctcggatctccggaggcctccggagcaggggggccagccgcggaccgacatggccttgattcacggtcatctcccggggaggatggttctccggagtgctgcagcccttcggaggctagcgtcccttgatccggagatactcccctaacaccttcttattcaaccaagctaggaccaatgaccatgacccttttggatatcaagatgaccacaggtttagatatcctcttaatcagccaaccgttttccagtcagaaaatcaagtgtgccaacaagttgcggaccaagaatgttggtggttggtcaggctacatcactttttcttatatctgtctaagtccatgagctaaaccaaaaacatctctcgctcttgctgtcttagcttgcaccgatattctctgcatctcggcacgatgatatcactcccattactgctcttcgtcctcttcttctctgcgctgctgctcttcccttcgagcagtgacgacgacggtggtggtgatgctgccggcgacgaactcgcgctgctctctttcaagtcatccctgctataccaggggggccagtcgctggcatcttggaacacgtccggccatggccagcactgcacatgggtgggtgtcgtgtgcggccgccggcacccacacagggtggtgaagctgcggctgcgctcctccaacctggccgggatcatctcgccgtcgctgggcaacctatccttcctcaggacgctgcaactcagcgacaaccacctgtccggcaagataccccaggagctcagccgtctcagcaggctccagcaactggtactgaatttcaacagcctatcgggtgagattccagctgctttgggcaatctaaccagtctctcggttcttgagctgactaacaatacactgtccggagcaatcccttcatctctgggcaaactcacaggtctcactgatcttgcactggctgaaaatacgctgtctggttccatcccatcatctttcggccaattgcgcagattatctttccttagcttagcctttaacaatttaagtggagcgatcccagatcctatttggaacatctcctctctcaccatattcgaagtcatatccaacaagctaagtggtacactgcctacaaatgcattcagtaatcttcctagtctgcaggaggtatacatgtattacaaccagtttcatggtcgtatcccggcatcgataggtaatgcttccaacatctcaatatttaccattggtttaaactcttttagcggtgttgttccaccggagattggaaggatgagaaatcttcagagactagagcttccagaaactctttcggaagctgaagaaacaaatgattggaaattcatgacggcattgacaaattgctccaatcttcaagaagtggaactgggaggttgtaaatttggtggagtcctccctgattctgtttccaatctttcctcttcgcttgtatctctctccattagagataacaaaatttcagggagcttacctagagatatcggtaatctcgttaatttacaatatctttctctcgctaacaactccttgacaggatcccttccctcttccttcagcaagcttaaaaatttacgtcgtctcactgtagataacaacaagttaattggttctctcccattgaccatcggtaatcttacacaactaactaatatggaggtccaatttaatgccttcggtggtacaataccaagcacacttggaaacctgaccaagctgtttcaaataaatcttggccacaataactttatagggcaaattcccattgaaatatttagcattcccgcactctctgaaattttggatgtgtcccataataacttggagggatcaataccaaaagaaatagggaaacttaaaaatattgtcgaattccatgctgattcgaacaaattatcgggtgagaaccctagcaccattggtgaatgccaacttctgcagcatcttttcctgcaaaacaatttcttaaatggtagcatcccaatagctctgactcagttgaaaggtctggacacacttgatctctcaggtaacaatttgtcaggtcagatacctatgtccttaggggacatgcctcttctccactcgctgaacctttcgttcaacagcttccacggtgaagtgccaaccaatggtgtttttgcaaatgcttctgaaatttacatccaaggcaatgcccatatttgcggtggcatacctgaactacatcttccgacgtgttccttaaaatcaagaaagaaaaagaaacatcaaattctgctgttagtggttgttatctgtctcgtttcgacacttgccgtcttttcgttactctacatgcttctaacctgtcataagagaagaaagaaagaagtccctgcaacgacatccatgcaaggccacccaatgatcacttacaagcagctggtaaaagcaacggatggtttttcgtccagccatttgttgggttctggatcttttggctctgtttacaaaggagaatttgatagtcaagatggtgaaatcacaagtcttgttgccgtgagggtactaaagctggaaactccaaaggcactcaagagtttcacggccgaatgcgaaacactgcgaaatactcgacaccggaatcttgtcaagatagttacgatttgctcgagcatcgataacagagggaatgatttcaaagcaattgtgtatgacttcatgcccaatggcagtctggaagattggctacaccctgaaacaaatgatcaagcagagcaaaggcacttgactctgcatcagagagtgtcacgccggaatttctatccaaaattccaaacgcttacatgtgtgtgaaccctcgtccaggaatcagccgagacacacaataacaaattgataatagagtacaattattactctaattaataagcgtataaaatgtcattacagaggtagatagttcctctcaatcaataaagatctaagcagcggaaaaataagataaacggcgcagacggctccactccacaggcagcttgaccaaggctacacctaatcctccacaccatcagcttcactgtagaactcttcctctgatgaatgattgcaaggtgagtatatgacatactcagcaagccacgcagcaaatatgcaagtgcacaggataacaaaggatggcatagtagggttttatttgcaaaagcagcatttagcaaacatttgagaatttaataaaacagttaagtaattaaacaatattaatccaacgctatacaacataccctgttgtataggcccaaccattctgaacaaccatcccggctgcacagttctatctccaaaccaggaatataccattccaaaccaggagctaatcaaattattaccaattaaagcaccttttattatgatgagaagggtgagactaatcacgaaagatattgttagacccgcctataaccgcgggcacggctattcgaatagttttactctgatcagaggtgtaccactgtacccacaagacacaaccccacatcatgtcaccatgtgcctcaataccaccacggtacctcggaaaggagttgtgacaataccccttgcataacacaatccactgcagtgcaccttcctggatcataatcacccccttaaaaacaaggcatggactccccagcgaccctcgtgggcttatctccgccacttctcagtctggtgccccgcaatgaaccatgctatataaaagataaagccgttgcccatgctggcttatggttggcacggttaatgtttcacaaccgaaactcgtgaaccggtccttaattgtcatgagcacgaccatcaaaaccatgtgctcacaacccaccattatcaggttttagttggcaaataattaattaacaaatcacgattgaccatcgtgaactatcattaagccatcattaaataacagtgagtcataagttatcccaatagtaagctaatgtttctaagcagggctaagcaattatatctaatatctagttgaaccaatatataaagctcactagtcaaattataataacccaaggtatcaaggaataaagtaatcaataacaaaagggctataacaaacaataggttaattccacccaatgacattcgaaaataaatgcaatatttgaatagaaacaatagctttaaataggatcaacatgctcaaagggttgtatgggatctgtgtgacttgccttgctggccttggaactcttcaaactcttctccggcgaaaacggactctccggaaacgacggaatctaaacaaaaagaagcaaaaccaccaaaacagcacataaaccaactaaatcggagctaagatgaattagttatgaatttttgaagattaaatcggattaaaacacttaaattgattttaattgaattatgacgcaataatgaattatttttgaaaaggaaaaggaggattattgcgtcagcgggctagggtttcggtggaccgggcacacaggcgacggctcacgcgaacggacggccgagatcgacttgatccaaaacggacggccgagatcgaacggtccacgaccggctcacagcgaacggccccgatgacgtcggcgatgacgtcaccaccggcggcggcggctcggcggctcgggcttgcacgctcgccggcgaacgacggcacggcggcgcgaatggaaggcaccaacgggtagagcgcgacgcggcgaactcaccggtgaccaaaagagcggcggaagatcaatggacggcgacggcgacgaggaggaagcggcggcaaacttcgggtcgacggtggcgacggtgctccggcggtcttcggcggcggcaaaggagcggacgagaacggcggcgacttggcgatcacgacggtggccttcccgagcgatgatgacgaccgagacggcggcgacgcacggctggagcgacggctacgacggcggcgctaggttgcacggcgctagagctcttccggcgacgagaggcgaaggcgaaggtggcgacgggtagaggagacaccggggaaccttttaaaggggctcgcaggcgacggcgaaggcccacggcggctggcgacgagaaggaaggtttagggttcggaggagggagacgaatccgattcgaactcgattccaacgatttccaaaacgaattagccgatgtttccaaaagagaaaaggtagaggagatcccggagattgtttcccctctatcaattcggccggaaacggaaaggatcgatcgaatttggaagggaacggcggcggcgcgaaactagggtttcgggcggcggcggccggaggttgacgacgaccctgacaggttggccccacctgtcagcgggcggacgcgcgcgcgcggcggcggactgggccggactgggccgaggagagagagagcggttttgggccgactttcggcccaaagccaaaagagactttttaaaacctttttcaatttaaattattcatgaaatgtaattccatttattaaaaatacttccttagctcaaataaatcccagaaaaatctaggaattatagaattaagcaaagtatttaatgaaattttatctggccccattttatattgtaatttattaatttaaaattagatcttctcttctaggcttttaaaataaattctaaaaattccatttaaacaacaatttatatattttgaattttcagggtgtgacagagagtgcccatactacgtgatgttgcatgtgcattggaccatcttcacttccatggccctgaccctattgtacactgtgatattaaatcaagcaatgtgttgttagatgctgatatggtagcccatgttggagactttggacttgcaagaatacttattgagggaagctcattgatgcaacagtcaacaagttcgatgggaatcagggggacaattggttacgcagcaccaggttaatcctaaactgtttatgtctacctcctttcattgttttttttagatttgctctggtccaacaaaaaatacctaaagatacagatacttgtacctcacagtactaaatagttttcgatcattgcattgttagatccaacgatcaggaaacgatttggtaccgtgaccgtgaggtatcggaatctcgagatattttttgttcgaccgtagcaaatctatttttttgtttgttttcttctctttaatgttttatgactatgaaataatttttatttctggaaaacagagtatggtgtcgggaacactgcctcgacacatggagatatttacagttatggaattctagtgttggaaacagtaaccgggctgcggccggcagatagtacattcagacctggcttgagcctccgtcagtacgttgaaccgggtctacatggtagactgatggatgttgttgacaggcagcttggtttggcttccgagacatggcttcaggctcgagatgtttcgccatgcagcagtattactgactgccttgtttcactgcttagacttgggctgtcttgctctcaggaattgccatcgagtagaacgcaagccggagatgtcatcaatgaactgcgtgccatcacagcgtctctctcgatgtcatccgacatgtgaagatgtgagacatgctgatgttatgtccgagtatttcgttgtaatgtaatgtgaagggtgagtgtgtgactgcttggttgtaagctatttcctgatctgcccatcagatcatgtatctgttctattgttgtatttctcagaacaactacacaccctaagtaggagtacacaatagtgtatttgtgtgatttcaatattgatgcatacccatgctatgtgctaaaattatatactgaaattttgagatgtctgaagttaacagtcaatcggggagcgattcacaccataccgcgaaatcgacctaatcagctaatctaattctacaggctgcctttgcatgacagtgtgatattaaattagcccagccctttttagcaaacgatgggagggtcaatgctctaga

SEQ ID NO: 9 - protein, 813 aa
MISLPLLLFVLFFSALLLFPSSSDDDGGGDAAGDELALLSFKSSLLYQGGQSLASWNTSGHGQHCTWVGVVCGRRHPHRVVKLRLRSSNLAGIISPSLGNLSFLRTLQLSDNHLSGKIPQELSRLSRLQQLVLNFNSLSGEIPAALGNLTSLSVLELTNNTLSGAIPSSLGKLTGLTDLALAENTLSGSIPSSFGQLRRLSFLSLAFNNLSGAIPDPIWNISSLTIFEVISNKLSGTLPTNAFSNLPSLQEVYMYYNQFHGRIPASIGNASNISIFTIGLNSFSGVVPPEIGRMRNLQRLELPETLSEAEETNDWKFMTALTNCSNLQEVELGGCKFGGVLPDSVSNLSSSLVSLSIRDNKISGSLPRDIGNLVNLQYLSLANNSLTGSLPSSFSKLKNLRRLTVDNNKLIGSLPLTIGNLTQLTNMEVQFNAFGGTIPSTLGNLTKLFQINLGHNNFIGQIPIEIFSIPALSEILDVSHNNLEGSIPKEIGKLKNIVEFHADSNKLSGENPSTIGECQLLQHLFLQNNFLNGSIPIALTQLKGLDTLDLSGNNLSGQIPMSLGDMPLLHSLNLSFNSFHGEVPTNGVFANASEIYIQGNAHICGGIPELHLPTCSLKSRKKKKHQILLLVVVICLVSTLAVFSLLYMLLTCHKRRKKEVPATTSMQGHPMITYKQLVKATDGFSSSHLLGSGSFGSVYKGEFDSQDGEITSLVAVRVLKLETPKALKSFTAECETLRNTRHRNLVKIVTICSSIDNRGNDFKAIVYDFMPNGSLEDWLHPETNDQAEQRHLTLHQRVSRRNFYPKFQTLTCV

SEQ ID NO: 10 - DNA (genomic), 5940 bp, Oryza longistaminata; single-stranded, linear
  CDS 2151..5855: /product= “receptor kinase-like protein” /note= “Xa21 family member A2; truncated by two mutations”
  mutation replace(2501..2503, “acc”): /note= “mutation compared to family member A1”
  mutation replace(4355..4356, “ac”): /note= “mutation compared to family member A1”
  misc_feature 5453..5697: /note= “Tourist-Ol2, transposon-like element”
  original source strain: IRBB21
  chromosome/segment: 11
  map position: 11q, RG103
tctagaaattgaattttaaaattgaatataatagagaaatacaactgaacataaatattttgtcattttcattccatttacatgtttttaaatctaaatttacagtgcattttgtatgtaattctaatacaaatttactgtaatttgtatttttagtcattttttcagtttctaaaagtacagtgcacttcagtgacctccttagtaagctatggggggaaaatggatatgcacatctgatctatctatgaaacatagaagaaatattcatcaccatgtactactttttatgaatgtttgttggccagataggatctgaaagatttctagtacagggttgtttcatgtattgtttcagataggatctgaaataaccactgtacagggttgtttcttgtatggtttccgcatatttaaacttttggttgcagatgtatggctaatcaattatgcagttttatggcaaacaaaaagggtggcaatagagtatatttcatcatgtggtatatgattgttttttgttgattagaccagaatagttgtgtgtatttgtttttttgttctgagaactattgtctgttctttattttcattcagacaatatgaatgctgttttgtacagattctttttgttgtgatgttcagacaatatgaatcagtggctgtttgtctgtttttcattcaaaattttcagagtgcacaagtgtgtgttgttaatccagaaattttcagaggattgcttgttctgttctgacctgaactagtaaaaaaacaaattcagatttggttcaatattcagattcttgtttttgtaatcattgctgtgcagaatctgtttgtactcctggtcatttagatttttagtctgtgtgtttgttgttcactgagtatttccattgggggcgagagtctcttttcttctatgcttttatacagaggttccaaatcaattgcagtgttgtgcaatattcagtgttgttcagatctatgtatatagcttctgttctattataatgcctgcagtaatctgctcatgtaaataacagaaaccctgtgggtattttgcatatttaggtacaaaattcaaagtttgcagaaacaaatctgttgagcattatgtcttctaaaccactaaacagttttttttctgtgctgatgttttttttctgccctttatgtgaatgtttacttctctctaatctctgtttggttttaattcatcagaggagctattttgcaagaataaatgtcaaagaacaacatgaagaaaaggaagactcaaaaatatgctgcgggtcacaggtaccttttgatttgtgattttcttgaattttgatccccaatatttcttgttttgagtctgatctggaagaatcttgcacttttgatccttctccaattctgagtttcttttccttcatctccatctttttccccagttttctttcttctcttcgatcgatctgagtttacctcttcttctctgtgtttgtctggaagaattattttgcctgttttgattactcgtcacgtgaatatgtcagcgatcttcttgcattgcaagttttgttgtcacgtgggtttgctcccttctttttgggcttcatttgggcttttttttgagctcataatctgattttttttttcttttcttgtttactaattgagcctaagttgggccctgaaagattcgttttttagttctgttggtgtcaaaaatctgggctgttggatcaaaatctgttggctgaaaaattcgacagatttggaactctattttctgttgactgaaagattagttttttagttctgctggagtcaaagtctgggctgttggatcaaaatctgttggctgaaaaattcgacagatttggaactctattttctgttgactgaaagattagttttttagttctgctggagtcaaagtctgggctgttggatcaaaatctgttggctgaaaaattcaacagattaggaactctattttctgtcgactgaacagtagacagagttaagcagaaacgaatatcacaattgctatgttcattgtcttgcgtgagcgctttttcttctatctgtctgtctagtgcatgagctaaaccaaacatctctcgctcttgcaccaatattctctgcatctctgcacaatgatatcactcccgttattgctcttcgtcctgttgttctctgcgctgctgctctgcccttcgagcagcgatgatggtgatgctgccggcgacgaacttgcgctgctctctttcaagtcatccctgcgataccaggggggcttgtcgctggcatcttggaacacgtccggccacggccagcagcactgcacatgggtgggtgttgtgtgcggccgccggcacccacacagggtggtcgagctgcggctgaactcgtccgacctgtccgggatcatctcgccgtcgctgggcaacctgtccttcctcaggacgctggacctcagcgacaaccacctgtccggcaagataccctaggaactcagcagtctcagcaggctccaacaactggtactgaatttcaacagcctatcgggtgagattccagctgctttgggcaatctaaccagtctctcggttcttgagctgactaacaatacactgtctggagcaatcccttcatctctgggcaaactcaccagcctcactgatcttgcactggctgaaaatatgctgtctagttccatcccttcatctttcggccaattgcgcagattatctttccttagcttagcctttaacaatttaagtggagcgatcccagatcctatttggaacatctcctctctcaccatattcgaagtcatatccaacaagctaagtggtacactgcctacaaatgcattcagtaatcttcctagtctgcaggaggtatacatgtattacaaccagtttcatggtcgtatcccggcatcgataggtaatgcttccaacatctcaatatttaccattggttttaactcttttagcggtgttgttccaccggagattggaagcatgagaaatcttcagagactagagcttccagaaactcttttggaagctaaagaaacaaatgattggaaattcatgacggcattgacaaattgctccaatctacaagaagtggaactgggaggttgtaaatttggtggagtcctccctgattctgtttccaatctttcctcttcgcttgtatctctctccattagagataacaaaatttcagggagcttacctagagatatcggtaatctcgttaatttacaatatctttctctcgctaataactccttgacaggatcccttccctcttccttcagcaagcttaaaaatttacgtcgtctcactgtagataacaacaagttaattggttctctcccattgactatcggtaatcttacacaactaactaatatggaggtccaatttaatgccttcggtggtacaataccaagcacacttggaaacctgaccaagctgtttcaaataaatcttggccacaataactttatagggcaaattcccattgaaatatttagcattcccgcactctctgaaattttggatgtgtcccataataacttggagggatcaataccaaaagaaatagggaaacttaaaaatattgtcgaattccatgctgattcgaaaaaattatcgggtgagatccctagcaccattggtgaatgccaacttctgcagcatcttttcctgcaaaacaatttcttaaatggtagcatcccaatagctctgactcagttgaaaggtctggacacacttgatctctcaggtaacaatttgtcaggtcagatacctatgtccttaggggacatgcctctgctccactcgctgaacctttcgttcaacagcttccacggtgaagtgccaaccaatggtgtttttgcaaatgcttctgaaatttacatccaaggcaatgcccttatttgcggtggcatacctgaactacatcttccgacgtgttccttaaaatcaagaaagaaaaagaaacatcaaattctgctgttagtggttgttatctgtctcgtttcgacacttgccgtattttcgctactctacatgcttctaacctgtcataagagaataaagaaagaagtccctacaacgacatccatgcaaggccacccaatgatcacttataagcagctggtaaaagcaacagatggtttttcgtcaaccaatttggtgggctctggatcgtttggctctgtttacagaggagaatttgatagccaagatggtgaaagcccaagacttgtcgccgtgaaggtactaaagctggaaactccaaaggcactcaagagtttcacggccgaatgcgaaacactgtgaaacactcgacaccgcaatcttgtcaagatagttacaatttgctcgagcatcgataacagagggaatgatttcaaagcaattgtgtatgacttcatgcccaatggcaatctggaagattggctacaccctgaaacaaatgatcaagcagagcaaaggcacttgactctgcatcagagagtgaccatactacttgatgttgcctgtgcattgcactatcttcaccgccatggccctgaacctgttgtacactgcgatattaaatcaagcaatgtgctgttagatgctgatatggtagcccatgttggagactttggacttgcaagagtacttattgagggaagctcattgatgcaacagtcaacaagttcgatggggataaggggaacaattggttacgcagcaccaggttaagtctaaactgtttatgtctacttcctataatcttctcttttttgaggtttcttctctctagtgttttatgactatgaaatattttttgctactggaaaacaaagtatggtgtcgggaacactgcctcgacacctggagatatttacagttatggaattctagtgttgaaaacagtaaccgggaagcggccgacagatagtacattcagaactggattgagcctccgtcagtacgttgaaccgggtctacatggtagactaatggatgttgttgacaggaagcttggtttggattccgagaaatggcttcaggctcgagatgtttcgccatgcagcagtattagtgaatgccttgtttcactgcttagacttgggttgtcttgctctcaggaattgccatcgagtagaatgcaagccggagatgtcatcaatgaactgcgtgccatcaaagagtccctctcgatgtcatccggcatgtgaagatgttggagtatttcattgtaatgtgatgtgtctatcagtacccttcacaactgatttcattctgccgtggtatttagttatttacaagagagtcactgaagggtgagtgtgtgactgcttggttgtagctatttcctgatctgcccatcagatcatgtatctgttctattgttgtatttctcataataaccacacacctaagggagggttcggcagaggagattgtgagttagtttgttttgttttccacgcgcacgcttcccgaactactaaacggtgtgttttttgcaaaaaaatttctatatgaaagttgctttaaaaaatcatattaatccatttttgaagtttaaaatagtttatactcaattaatcatgtactaatggctcacctcgttttgtgtatcttcccaatcttctcttttcccctcctctcaaactcaccctaagtacacaacactgtatttgtgtgatttcaatattgatgcatatatacccatgctatatgctagaattatatacaaaaattttgagatgtctgaagttaacaatcaatcagggagcgattcacaccaaaccgcgaaatcgacctaatgagctaatctaattgtacaggctgcctttgcatgacagtgcgatattaaataagcccagccctttttagcaaaggatgggagggtcaatgttctaga

SEQ ID NO: 11 - DNA (genomic) 5147..5547), 7204 bp; single-stranded, linear
  CDS join(1683..1758, 1771..2186, 2347..4352,: /product= “receptor kinase-like protein” /note= “Xa21 gene family member F”
  misc_feature 233..384: /note= “Gaigin-Ol1, transposon-like element”
  misc_feature 404..532: /note= “Gaigin-Ol2, transposon-like element”
  misc_feature 1080..1239: /note= “Tourist-Ol1, transposon-like element”
  misc_feature 6201..6583: /note= “Crackle, transposon-like element”
  misc_feature 6750..6956: /note= “Ds-rice3, transposon-like element”
aagctttctaaattatttaactctaagtctgttattatccccaagtacatcatcatcatacataatatttcatattcacgacatccttaagctagatgcttttggccattctcttatctttttaaagaaattctctcccaattaagatgagagtgtcttctagcaatttgccagtttttacaatgtctttgagtcctcacacattttcatgatgttaccaataaattacgaacgccgtgtttagttctaaagtttttcttcaaacttacaacttttcaatcgcatcaaaactttctcctacacacacaaactttcaacttttccatcacatcgttccaatttcaaccaaacttccaattttggtatgaactaaacacagccgaaaacaaaatttgtgtgttatggccctgtttagattctaacttttccattacatcaaactttcctacatacacgaactttcaacttttccgtcacatcgtttcaattttttaaaacttccaattttaacgtggaactaaacacaacctatataacgaaatttgtcaaaaacttaatggtgaaagtcacacctcaaaggaagggcgcgcctctagtcaagaacatcaattaaaaaggtacacaggttgtactagcttgttcatgtttaatcttgcgtctgcgagacgctaaatccatgccaaacaaaagtgcttctatagagataatcataagaatatggtttgggaccatatccaactgctcagaagaatctcgttcggaggtgaaggttaagatgttcacctctccacacataaaacaaagcgatctttttcgcataattaattaagcattagataaaataaacttaaaaaataaatcaatatgatttttttagaaaaaaaatatatacactaagtataagcattgtcaaggaggaagaaacacacacttccatatagagagatagaaacatagctataggtagtgtcactgagtatttttcatcacgcatatgcatataaaattagggggtgtttacatccataggtgtaaagttttggcatgttatatcgagtattacgtagaatgtcgtattaggtgttcgggcactaataaaaaaataattacagaatccgttagtaaaccgcgagataaatttattaagcctaattaatccatcattaacaaatgtttaccgtagcaccacattgtcaaatcatggagcaattaggtttaaaagattcgtctcgcaaattagtcataatctgtgcaattagttatttttagactatatttaagacttcgtacaggtgttcaaacgttcgatgtgacatggtgcaaaattttagggtgtcatctagacactcccttaattagaaagttaggaagaggcggtaaagaacgcagcatgactgaaactttgaaaatttgataaggtacaccaactggagtatcttttattttcattgaagactttgaccagaagagcttgacccgtttttcttggagtagccagtaatgtttcattcttttccttttgctgggacttctttttattttttttgacaggagccatttgttgggacttgggatccctttactgttataggaccagtgcttgaatccaaacactgcattgatcagctcagctcattgtagcgcactcctccgcatgcatggcgagatcaccaacgtcggtcatgatctcttctttgctgctgctgctgttgatcggcccagcgagcagtgacgatgatgctgctgctgctgctgctcgtaccagtacaggcggcgtcgccggcgacgaactcgcgctgctctctttcaagtcatccctgctacaccaggggggcttgtcgctggcatcttggaacacgtccggccacggccagcactgcacatgggtgggtgttgtgtgcggccgccgccgccgccggcacccacacagggtggtgaagctgctgctgcgctcgtccaacctgtccgggatcatctcgccgtcgctgggcaacctgtccttcctcagggagctggacctcagcgacaactacctctccggcgagataccaccggagctcagccgtctcagcaggcttcagctgctggagctgagcggtaactccatccaagggagcatccccgcggccattggagcatgcaccaagttgacatcgctagacctcagccacaaccaactgagattggtgccagcttgaaacatctctcgaatttgtaccttcacaccaatggtttgtcaggagagattccatctgctttgggcaatctcactagccttcagtattttgatttgagctgcaacagattatcaggagctataccttcatcgctagggcagctcagcagcagtctattgactatgaatttgcgacagaacaatctaagtgggatgatccccaattctatctggaacctttcgtctctaagagcgtttagtgtcagcgaaaacaagctaggtggtatgatccctacaaatgcattcaaaacccttcacctcctcgaggtgatagatatggacactaaccgtttccatggcaaaatccctgcctcagttgctaatgcttctcatctgacacggcttcagattgatggcaacttgttcagtggaattatcacctcggggtttggaaggttaagaaatctcacaacactgtatctctggagaaatttgtttcaaactagagaacaagaagattgggggttcatttctgacctaacaaattgctccaaattacaaacattggacttgggagaaaataacctggggggagttcttcctaattcgttttccaatctttccacttcgcttagttttcttgcacttgatttgaataagatcacaggaagcattccaaaggatattggcaatcttattggcttacaacatctctatctctgcaacaacaatttcagagggtcacttccatcatcgttgggcaggcttagaaacttaggcattctagtcgcctacgaaaacaacttgagcggttcgatcccattggccataggaaatcttactgaacttaatatcttactgctcggcaccaacaaattcagtggttggataccatacacactctcaaacctcacaaacttgttgtcattaggcctttcaactaataaccttagtggtccaatacccagtgaattattcaatattcaaacactatcaataatgatcaatgtatcaaaaaataacttggagggatcaataccacaagaaatagggcatctcaaaaatctagtagaatttcatgcagaatcgaatagattatcaggtaaaatccctaacacgcttggtgattgccagctcttacggtatctttatctgcaaaataatttgttatctggtagcatcccatcagccttgggtcagctgaaaggtctcgaaactcttgatctctcaagcaacaatttgtcaggccagatacccacatccttagcagatattactatgcttcattccttgaacctttctttcaacagctttgtgggggaagtgccaaccattggtgctttcgcagatgcatccgggatctcaatccaaggcaatgccaaactctgtggtggaatacctgatctacatctgcctcgatgttgtccattactagagaacagaaagcattttccagctctacctatttctgtttctctggtcgcagcactggccatcctctcatcactctacttgcttataacctggaacaagagaactaaaaagggagccccttcaagaacttccatgaaaggccacccattggtctcttattcgcagttggtaaaagcaacagatggtttcgcgccgaccaatttgttgggttctggatcatttggctcagtatacaaaggaaagcttaatatccaagatcatgttgcagtgaaggtactaaagcttgaaaatcctaaggcactcaagagtttcactgccgaatgtgaagcactacgaaatatgcgacatcgaaatcttgtcaagatagttacaatttgctcgagcattgataacagagggaacgatttcaaagcaattgtgtatgacttcatgcccaacggcagtctggaagattggatacaccctgaaacaaatgatcaagcagaccagaggcacttgaatctgcatcgaagagtgaccatactacttgatgttgcctgtgcattggactatcttcaccgccatggccctgaacctgttgtacactgtgatgttaaatcaagcaatgtgctgttagattctgatatggtagcgcatgttggagattctgggcttgcaagaatacttgttgatgggacctcattgatacaacagtcaacaagctcgatgggatttagagggacaattggctatgcagcaccaggtcagcaagtccttccagtattttgcattttctgatctctagtgctatatgaaatagtttttacctctagtgaaactgatggagaatataagtaattaattgaactaattaaattgcacaaaaataagattatttgccatatctattcagatgctaaatatagctagttcatagaggtacatattttttttatataggaatctagagctactacacactcaaatcaaattatgggtgttttctgctctacactgcaatatgaaatgattatcagaaggatcaaatttgagtaaatttgtcaattctacatttaagaaacacttttttttgtatgtactagttattacaattttttatttcaagaacttgcattgaccatgaaaagtacttggtactacttctaattcccacatggaggtggtgaaaataatatagatacaaaaacgaagtatcatatgttgtgtgatatactataatcacaatgaacacaaacaggattcgtacaaaagtaattggccatcatagcaactgattgcttggggtaactgtatagcacaatcataccaaatttctttagatatgtatttgtaaattagattcttaaagttaaatatgaaatttcattggtatttatgtttctttatataataaaaattaatccaacctttacatctaccatttgtccagccatccttgttatttgtgatatttaacacgtaattttacataattatacatccaagttctttttatttaacactggaaatttgaaatcgtatttcctactcaaacagagtatggcgtcgggcacattgcatcaacacatggagatatttacagctatggaattctagtgctggaaatagtaaccgggaagcggccaactgacagtacattcagacccgatttgggcctccgtcagtacgttgaactgggcctacatggcagagtgacggatgttgttgacacgaagctcattttggattctgagaactggctgaacagtacaaataattctccatgtagaagaatcactgaatgcattgtttcgctgcttagacttgggttgtcttgctctcaggatttgccattgagtagaacgccaaccggagatatcatcgacgaactgaatgccatcaaacagaatctctccggattgtttccagtgtgtgaaggtgcgagcctcgaattctgatgttatgtcttgtaatgttttattgccactagtcttcagattggaatgctcttccgatcagacttcttcagtggtatctaccacacgatcactaaagtcatcgtggctatttcctgatccagcatatctgatcatgcatgttctgtgttttatacctgtattttactctgaattgccacacctcaaccctgcctctgtttgtttggcatacaaaagatagtgatgagtatattgtttcaggggcttcctagttggcgtgtgtgcttaccggcacgcacgcagcccgagggtgggtttctttttttttccattgttattccgttgcttttttccaccacggtagattttttttttctggatttccattttttccgttgtttttctctatcgcttatgctggcggatttttttccgtggtttttttttcaagacgagtatatctaatgtaactaacatgttacttttagataacgatggttattaagataagatttttttctggaagatttttgtaagtaaatggtaaaaaatatggaaatggaaacggaaatagttttgctgttataccgatcgtttccatatttaccgtattcttatagaaattaccgtttcttataatatggtaattaccgtatttctaaatatgttgatatcgattttgctatatattgcgacaaattttctcccaaaaatttgatagatgtaattatagtacaatcgtagtgtaattacactgtaactatagtgtaacttgtatgtaactttcaaaaatctctctgtaatatgttattttggtaaaatagaggttgtgggaacaaatccttacacatatgtgtgcgctgtgattttctttcttcctcaccaaaacaaaacttataatagatttaacaattcaaaattacgtgaaacttatacaagttacaccgtagttacatgcaagttacagtgtaattacactacgattgtactataattacatctgtcaaatttttaggagaaaatttgtcgacaaatatataggtgatcccgttgatatttatagggtatgtctctacttgactcacagtttagagattgattgactatttaatcaaatccctaacttgattgcacggctaaaatggagttgatttctaatttatatagtatagattgaatttattcgtacatataacatacttatgtaaagttaaatatatgttttctatagtttaatgtttctgtatttgttaccggttttcgatctataccgaccatgtttccttcagtattattccgtttccggttttctgatatttctgatatcgttttcgtttccgagtttaccgttttcgatttcatttccgagaaaaatatgattatggaaatggttgaggctgttttccgatcgtttccgaccgttttcttccctacccgtagcaataatatataatattttatctctaatctttctctctctcatatcaacgaatattcgctaagagactgctattaacaaggcttttatatatatatatgtacatatatatatatatatatatatatatatagacacacacacatacatacatacatacatacatatatatacatacatatacatatatatatatgtatacatacatatacatatatatatatgtatacatacatatac

SEQ ID NO: 12 - DNA (genomic), 1332 bp, Oryza longistaminata; single-stranded, linear
  - 1..1332: /note= “3′ flanking sequence of Xa21 gene family member F”
  original source strain: IRBB21
  chromosome/segment: 11
  map position: 11q, RG103
gccgtcgatcaatcatttggaaacggcccacatcttttccatctatatgcattcatgaaatacatggtatatcccatcgatcggacatcacctgttagcgcgtacgccatcgtcgtgatcaacctagctagggcaaacgtcctccgatcgccaccatcaccaatgaacaagctgctgcggcctctcggtggcctgaggttgctcaaccgagaagaacatccgttccgatgcttctcctcctccatcgatctcgtcttcccaggtcgccgccgccgccacatggcaaccaccgtgacccacccgccgccgacggaatcccgctggttcgacggcggcggccgcgactgctgacccggcctcggtgatgctggaacgttggggctgcctcaggggctccacgccggcgaacgtcgccgccgacgacaacaccgccgcggagtcccgcacctcccgcggccaacccctccgcgtcgccctcgcccgcgcgtcgccgccggcgatctccttcatctgcttcgatcgcggggatgatggctacgtcatcgcggctcacggcgactctgtcctcttccggatgagttggaacgactacttcgtctacatggccgccgccggcaagccgccgtcgctgacgctgctccccgtctgcgacatccccatgaacgagcgctgctgggtcagcaaggaccgtttcaacgacagcttccgcaccacgggccgggtgttcgaccagcaggacaccggcatcctgcgcctccgcggcggcgaggaggcgccgcctctagtggcgcagctccagatcgcgcacgaggcgccgttcgacacggccgagctctgcgtgctccgccccggccacggccacggcgagtgggagctcaagacggcggtgcccatcgtccaccacgacggcggcggcgaacgccgccatggcctggagatgtggcaggagacaacgtggccgtccccgtcggcgaccgcttcatgtgctgggccaactacgacctcgccaccttcctcatctgcgacatggcggcggcggatctcgacaaccccaagctcctgtacgttccgctgccggtgaaccagtgccacccaagggagagcgacttcgacgacgaccaccaccacgacgagctgattccatggggagtacttccgcaacatcgtcgccaccggcgccgacggcggcgacgacattgtgcgattcgtcagcatccacaaccgctgctgctgcggcgcgcccgtgatacacaacctgtgcgaacgctccagctcggcgttcatggtgaacatctggaagcttgcctgcggaacccccgcgcccgcgacagc

SEQ ID NO: 13 - DNA (genomic), 840 bp; single-stranded, linear
  CDS 1..840: /note= “Xa21 gene from cassava”
tcaagcaacaatttgtcaggtcaaatccctgaatttttggcagggtttagttttatatatcttaacttatctttcaataattttgaaggcagagtgccaacagatgggatattcaagaatgcaagcattgtttcagtcacaggaaactctaagctttgtggaggcatacctgagtttcaacagcctgcatgcaacttcaaaaggtctgaaaaaaggagagttaaggtaattgttggtattattgcaggaggtttaggagcaattttggtggtgttgtcctttatatttcttttgagattaagaaagaaaagtcacaaacccagttcatcctattcagaaaattcacttttggaacttccaaaagtgtcatatagagatctctataaggccactgatgggttctcctcagaaaatttaattggtactggtagttttgggtccgtatataaaggaattcttgatgaaggtggaccagttgttgctgttaaagtgcttaacctccagcatcatggagcagctaagtctttcatggctgaatgtgaagccttgagaaatatcagacaccggaatcttgtaaagatactaactgcttgttcaggtgttgattatcaaggcaatgatttcaaggcactggtttatgagtacatggataatggaaaccttgaggagtggttgcatctaccagtttcagcagatagaaatcatggggagcctaagaatctaaatcttcttcagagagtaaatattgcaattgatgttgcttctgcaattgaatatctccatcatcattgcggaaatccaatagttcattgtgaccttaaatcaagcaatgtgctgtta

SEQ ID NO: 14 - protein, 280 aa
SSNNLSGQIPEFLAGFSFIYLNLSFNNFEGRVPTDGIFKNASIVSVTGNSKLCGGIPEFQQPACNFKRSEKRRVKVIVGIIAGGLGAILVVLSFIFLLRLRKKSHKPSSSYSENSLLELPKVSYRDLYKATDGFSSENLIGTGSFGSVYKGILDEGGPVVAVKVLNLQHHGAAKSFMAECEALRNIRHRNLVKILTACSGVDYQGNDFKALVYEYMDNGNLEEWLHLPVSADRNHGEPKNLNLLQRVNIAIDVASAIEYLHHHCGNPIVHCDLKSSNVLL

SEQ ID NO: 15 - DNA (genomic), 2193 bp; single-stranded, linear
  - 1..2193: /note= “DT4 Xa21 gene from maize”
ggatccccgcgcagcttggccggtcgtcttcgctccagcgcgtgcgcctgggaagcaacatgctctccgggccgatcccgccgtcgcttggcggaatcgccacgctgaccctgctggatgtgtcaagcaacgagctcacggtggcatcccagcggcgctcgctcaatgcaggcagctcagcctcatcatcgtcctgagccacagccgcctgtcaggggcggttccgggctggctgggctcgctgccgcagctcggcgagctggcactctccaacaacgagttcaccggagcaatcccgatgcagctcagcaactgctccaagctcctgaagctgtccctcgacaacaaccagatcaatggaacagtgccgcctgaactcggcggcttggtgtccctcaacgtactgaatctcgcacacaaccagctctcaggtccgattccgacgacggtcgcaaagctgagcggcctctatgagcttaacctgtcgcagaattacctgtccggcccgatccctccggatatcggcaagttgcaagacttgcagagcctgctggacttgagcagcaacaatctcagtggccacatccctgcatcgctgcggctcactccccaagctggaaaacctgaacctgtcccacaacgctctggtcggcgcggtgccgtcccagctcgctggaatgagtagcttggtgcagctggacctgtccagcaccagctggaagggaagctgggcaccgagttcggccggtggccgcaggccgcattcgctgacaatacagggctctgcggtagccccttgagaggttgcagcagcagaaacagccattcggcgctgcacgcggcgaccatcgcgttggtgtctgcggtggtcacgctgttgattgtcctcctgatcattgcgattgcgctgatggtggtgcgccgcagggcccggggttcaggcgaggtgaactgcacggcgttcttgtcgtcgagctcgggcagcgcaaaccggcagctcgtcgtcaagggctcggcgcggcgggagttccggtgggaggcgatcatggaggccacggcgaacctgagcgaccagttcgccatcgggtccggcggatcaggcacggtgtacagggcggagctgtccactggcgagacggttgccgtgaagaggatagcgcacatggacagcgacatgctgctccacgacaagagcttcgcgcgggaggtcaagatcctgggccgcgtccgtcaccggcacctggtcaagctgctcggcttcgtcacgtcccgcgagtgcggcggcggcggcggcatgctcgtgtacgagcacatggagaacggcagcctctacgactggctgcacggcggcagcgatggccggaagaagcggacgctcagctgggaagcgcggctcatggttgccgccgggctggcgcagggcgtggagtatctccaccacgactgtgtgccccgcatcgtgcaccgggacatcaagtccagcaatgtgctcctcgacggcgacatggaggcgcacctcggcgacttcggcctcgccaaggccgtcgccgagaaccggcaggccgccttcgataaagactgcaccgagtcagcttccttcttcgccggatcatacgggtacatcgctccaggtaatttcgacggcaatctgaaatgctatagaaacgcagtagctcaggcgacgcggccagttactgacagtggacgtgccacattatctctgcagatgtgcttactccctggaggcgacggagagaagcgacgtctacagcatgggcatcgtggttatggagctcgtcaccgggctcttgccgaccgacaaaccttcggcggcgaacatgacatggtgaggtgggtgcagtcgaggatggacgcgccgttgccagcaagggagcaggtgttcgatcctgctctgaagccgctggcgccgcgtgaggagtcgtcgatgacggaggtgctggaagtggcgctccggtgcacaagggctgcttcacgtttcactcgattactgacgcgtctgcgagatgaattaggctttgttcgtttgtgtcggagtcgagtgtaatccaacacaaacaagaccttaggatttgaaaagtgggctggattgtcttagcagccgctcaaaattggatcgcttaatttcaaattttgagctcccttgtacaatcatcatttggaaatgtccagcag

SEQ ID NO: 16 - DNA (cDNA), 3045 bp; single-stranded, linear
  - 1..3045: /note= “DM4 Xa21 gene from maize cDNA clone”
ggcacgaggggaactagacatgtctgggaacaagattctgtcgggccgagtaccggagttcctggggggcttccgagcattgcggcgactcggacttgccgggaacaacttcaccgaggaaatcccggatgagctgagcctcctgtgcggaacgctggttcagctagacttgtcaagcaaccaattggttggcggtttgccagcgagcttctcaggatgcagatcgcttgaggtgctcgatctgggtagcaaccagctgtcaggtgactttgtgatcactgtgatcagcaagatctcttctctgcgtgtgctgaggctgccgttcaacaatatcacaggcacaaatcctctgcccacgctagcagctggctgccctttgcttgaagtcattgatctcgggtctaacatgctggaangagagatcatgcccgagctgtgttcatctttgccatcactcagaaagctgctcctacccaacaactacatcaatggaaccgtgccgccctcactcggcaactgcagtaatctggagtcactggacctcagcttcaacctcatggttggtccgatcaccctgaagtactgttgcttcctaaacttgttgaattggtcatgtgggcaaacantctctccggtgaaataccaaacacgcnatgctccacagcacaacactgaaaanccgtcntaaactacaacaacataaccgaatgatcccgttcncntcnccagtngctnaatccatatggtgtccttccggcaacacataacgggaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatcctntttcntnacagnggggatttttatatggtgtaattgcggcaacggatgacggggtntcccgcggtttgggaccttccagagtttgccatttacagtgcacaggaattcattttttgttcctgtgccagcagagnttgttcgctgcagcaccttatntggttgattttcaacagcaacaatttttccggtgcgataccgccgcagttagcagcaacaggcagggntcatcactggaggcatggttttctgggaagcagttcgcgttcctgaggaatgaggctggaacatctgcccaggtgctggagtgctgttcgagttctttgacatccgtccggagcggctggcccagttccctgctgtgcactcgtgtgcctccacgaggatatacactgggatgacagtgtacaccttcaaccaaagtgggagcatgatattccttgatctctcgtacaacagcctcacaggcacaattccggcgagcctggggaacatgacgtatcttgatgtccttaacctggggcataatgaccttaccggtgcaattccagatgcgttcacagggttgaaggcgattggtgtccttgacctntcgcacaatcacctcaccggtgtcatccctgctggactggggtgtttaaatttcctagctgacttcgacgtctccaacaacaacctcactggtgagatacccacgtcagggcagctcagtacatttccagcatcccgttttgagaacaactccggcatctgtggcatccccttggatccttgcacgcacaatgccagcactggaggtgttcctcaaaacccctctaacgtgcggaggaagtttctcgaagagttcgtgctccttgcagtgtcgctcaccgtgctcatggtggccaccttggttgtcactgcatacaagctcaggaggccccgtgggagcaaaactgaagagattcaaactgctgggtatagcgacagccccgcatcgtccaccagtacaagctggaagctttctggttccaaagagccactgagcatcaatctggcgatatttgagaatccgttgaggaaactaacatatgcccacctgcatgaggctaccaatggcttcagctcggaagctcttgttggcacaggaggattcggtgaggtttacaaggctaggctcatggatggcagcgttgtggctgtcaagaanctgatgcatttcacaggccaaggcgaccgggagttcactgcagagatggagaccattggcaagatcaaacatcgcaaccttgtgccgttgctaggctactgcaaagttggcgacgaacgtctgcttgtgtacgagtacatgaataatggaagcctggatgtcttgctccatgaaagggacaagactgatgtgggtcttgattgggcaacaaggaagaagattgcagttggctcggcaagaggactggccttcctccaccatagttgcatcccacacatcatacaccgggacatgaagtcaagcaacgtgcttcttgacgataatctcgatgcctacgtatcggatttcggaatggcgcggctcgtgaatgctgttgactcacatctaaccgtgagcaagctcttaggaacacctggttatgtggctcccgagtacttccagtcggttatttgcacaactaagggcgacgtctacagctatggcgttgttcttctggagcttctctcagggaaaaaaccaatcaatccgactgaattcggcgacaataatctcatcgactgggccaagcagatggttaaggaggaccggtgcagcgagatatttgatcctatattgaccgacacaaaatcctgcgagtcggagctgtaccagtatctggcgattgcttgccagtgcttggacgatcaacctagtcgcagacctacgatgatccaggtcatggcaatgttcagtgagtttcagattgactctggcagcttcttcttggacggcttctcgctcgattcagatagaggaatcatctgaaaaaaaatgtgtaaatgttattgatccctgcagattatatgattcactggatttaggtattagcttagccatgtttaactcatgttaacaggatacaaacagatgtaaatttgtttcggttgccgtacatagtacacaacagcttcaacacagataccatatagagttgtttccaaaaaaaaa

SEQ ID NO: 17 - DNA (genomic), 3293 bp; single-stranded, linear
  - 1..3293: /note= “TRK1 Xa21 gene from tomato”
  CDS 92..2974: /product= “TRK1”
atcgggcaggtcttcaaaatacttgttacatcttcttcttacctttgatattttccaaagtatttgtaacttcaaatcactagttatctaaatggctacttctaacacaagtctcttgtttttcgcgtatttcctccttgtgttccttattactccatctcaatcgcgtaacctgtctctgagacgacaggctaaaactctagtttcattgaaatatgcatttgtacaatcatctgttcctagtactctgtccaattggaacatgtcgaattatatgtctatatgttcttggacaggtataacgtgtgatgataccaaatcagtaacttccattgatatatccaatctaaacatttctggctctttatcacctgatattcatgagctcactagacttcgcgtcctgaatatttctaacaatttgtttagtggaaacttaagctgggagtatcgcgagtttaatgtacttcaagtgttggatgcttataacaacaatttctctggtccactccctttgggagttactcaacttgtgcagctcaagtacttgaatttcgggggtaactacttttcagggaagattcctttgagttatggtagttttaatcagcttgagttcctgtctcttgctgggaatgacttgcacggtcctataccgagggagctggggaacgttacgagcctcaggtggttacagttgggttattataatcaatttgatgaggggattccaccagagttggggaaacttgttaatttggttcatctagatctttcaagctgtaacttaacgggttcgattccaccagaattgggcaatcttaatatgttggacactcttttcttgcaaaagaatcaacttactggtgtatttcctcctcagctagggaatttgacaaggttaaaatctcttgatatctcggtcaatgaactcacaggagagatcccggttgacttgtcaggactcaaggagctcatattgttgaacctctttatcaacaatttgcacggtgagattccaggatgtatcgcggagctgccaaagttggaaatgttgaatctttggaggaataatttcactggctcgattccttctaagcttgggatgaacggtaaactaattgaaattgatctgtctagtaatagactcactggcttgataccaaaatctctatgctttgggaggaatttgaaaatcttgattcttcttgataattttctgtttggacctttacctgatgattttgggcagtgtcgaacgttgtccagagtcagaatgggacagaattacttgagtggatcaataccaacagggtttctttatttgcctgagttgtcactggtggaactgcagaacaactacatcagtggacaactctggaacgagaaaagctcagcgtcttctaaacttgaagggctgaacctgtcgaacaatcgcttgtctggtgcacttcctagtgctattggaaactattcagggctgaagaatcttgtgttaactggaaatggtttctcaggtgatatcccttctgatattggcagactaaagagcatcttaaagctggacctgagtagaaacaacttctctggcacaatccctcctcagattggtaactgtctttccttaacttacttggatttgagccaaaatcaactttctggtcctatcccagttcaaattgctcaaattcacatcttaaattacatcaatatttcctggaatcacttcaacgagagccttcccgcggagattggcttgatgaagagtttaacttcagcagatttttcccacaataacttatctggatcaatacctgaaacaggccaatatttatatttcaactcaacttccttcaccggcaacccttatctctctggatccgactcgactcctagcaacattacatccaactcaccgtcagaacttggagacggaagtgacagcagaactaaggttcctacaatatacaagttcatatttgcatttgggctcttattctgctccctcattttcgttgtcttagcaataatcaagacaagaaaggggagtaagaattcaaatttgtggaagctgacagcatttcagaagcttgagttcggaagtgaagacgtcttgcagtgcttgatagacaacaacgtcatagggagaggtggagcagggatagtgtataagggaactatgccaaatggtgatcatgtcgcggtgaagaaattgggaataagcaaaggctcacatgataacggcctatctgctgaacttaacacattagggaagatcaggcataggtacattgtgagactgctcgcgttttgttcaaacaaggaagtcaacttgctagtttatgagtacatgctaaatggaagcttaggtgaagtgcttcatgggaagaacggcgggcaactccaatgggaaactaggctaaaaatagccatagaagctgccaagggcctttcttatttgcaccacgattgctcccctatgataatccaccgcgatgtcaagtccaacaatatattgttgaactctgaacttgaagctcatgttgcagattttggattagccaagtactttcgtaacaatggtacctctgagtgcatgtctgcaattgcaggatcttatggctacattgctccagaatatgcatacacgctgaaaattgatgagaaaagcgatgtgtatagctttggagtggtgttgttggagcttataacaggacgaaggccagtaggaaattttggagaagaaggaatggacattgtacaatgggcgaaaacggagacaaaatggagcaaagaaggggtggtgaaaatcttggatgagaggctaaaaaatgttgcaattgttgaagctatgcaagtattttttgtagcaatgctttgtgttgaagagtacagcattgagaggcctacaatgagggaagtagtccaaatgctttctcaagctaaacaaccaaatactttccaaatccaataatctaattgtggctctacttattgtatgcttgggaacaccccttttgttagctttgcaaaagtgaaatcacaaattaatctaagtgaagtagttgcaaaattaatttgcaattatgttagatcttagggtatgatatctaactatatcctctcaacttggaatagtgtattggatgtgtagaactagtattagtatccgcgtgatgtgtggcgaatatcaaaagaaagtcgacrcakrmmrctaatycckcygwtwcwramgkmswccmggmskcgawykccrccratactgacggactccaggagtcgtcgccaccaat

SEQ ID NO: 18 - protein, 960 aa
MATSNTSLLFFAYFLLVFLITPSQSRNLSLRRQAKTLVSLKYAFVQSSVPSTLSNWNMSNYMSICSWTGITCDDTKSVTSIDISNLNISGSLSPDIHELTRLRVLNISNNLFSGNLSWEYREFNVLQVLDAYNNNFSGPLPLGVTQLVQLKYLNFGGNYFSGKIPLSYGSFNQLEFLSLAGNDLHGPIPRELGNVTSLRWLQLGYYNQFDEGIPPELGKLVNLVHLDLSSCNLTGSIPPELGNLNMLDTLFLQKNQLTGVFPPQLGNLTRLKSLDISVNELTGEIPVDLSGLKELILLNLFINNLHGEIPGCIAELPKLEMLNLWRNNFTGSIPSKLGMNGKLIEIDLSSNRLTGLIPKSLCFGRNLKILILLDNFLFGPLPDDFGQCRTLSRVRMGQNYLSGSIPTGFLYLPELSLVELQNNYISGQLWNEKSSASSKLEGLNLSNNRLSGALPSAIGNYSGLKNLVLTGNGFSGDIPSDIGRLKSILKLDLSRNNFSGTIPPQIGNCLSLTYLDLSQNQLSGPIPVQIAQIHILNYINISWNHFNESLPAEIGLMKSLTSADFSHNNLSGSIPETGQYLYFNSTSFTGNPYLSGSDSTPSNITSNSPSELGDGSDSRTKVPTIYKFIFAFGLLFCSLIFVVLAIIKTRKGSKNSNLWKLTAFQKLEFGSEDVLQCLIDNNVIGRGGAGIVYKGTMPNGDHVAVKKLGISKGSHDNGLSAELNTLGKIRHRYIVRLLAFCSNKEVNLLVYEYMLNGSLGEVLHGKNGGQLQWETRLKIAIEAAKGLSYLHHDCSPMIIHRDVKSNNILLNSELEAHVADFGLAKYFRNNGTSECMSAIAGSYGYIAPEYAYTLKIDEKSDVYSFGVVLLELITGRRPVGNFGEEGMDIVQWAKTETKWSKEGVVKILDERLKNVAIVEAMQVFFVAMLCVEEYSIERPTMREVVQMLSQAKQPNTFQIQ

SEQ ID NO: 19 - DNA (genomic), 3850 bp; single-stranded, linear
  - 1..3850: /note= “TRK2 Xa21 gene from tomato”
  CDS 2..3487: /product= “TRK2”
tgactctctctgtctctctctgttcgcagccccaaaaagtagggttagggctagggtttttgagtttcaaaaccccatttctggttcctataatcttcacatacaaggggagtttgtctctgttgcattctttgaagacccttttggggttttactaatgggtcgttgttgttttgtcatcaaatggtactatcatgacatacccttgaaagtttttctcatcctttgtgttttcttcttagttcatggctatgcactttcctcggattcggataaatcagcgctcttggagttaaaggcctcattttcagattcctctggagtgatttctagctggagctccagaaataatgatcactgttcatggtttggtgtctcctgtgattccgattcacgtgttgtggctttgaacatcactggaggtaatttgggttctttatcttgtgctaaaattgctcaatttcctttgtatggctttggaattacaagggtttgtgctaataatagtgtcaagcttgttggtaaagtacctctcgcaatatcaaaattaactgaactaagggttttatccttgccttttaatgaattgcgtggtgatattccattgggaatttgggatatggacaaacttgaagttttggatctgcaagggaatttaattactgggtctttgccattggagtttaaggggttgaggaaattgagggttttaaacttgggttttaatcagattgtgggtgccataccgaattccttgtcaaattgccttgctctacaaatctttaatcttgctggaaatagggtaaatgggaccattccagcattcattggtggatttgaagatctgaggggaatctacctgtcttttaatgagcttagcgggtctattcctggtgaaattgggcgttcttgtgagaagcttcaaagtctagagatggcaggtaatatcttaggtggtgttattccaaaaagtttagggaactgcacacggttgcagtcacttgtcttatattcaaatttgttggaagaggctattccagctgaatttggtcaactaactgagctcgagattcttgatttgtctaggaacagcctaagtggtcgactaccatctgagctgggaaactgctcgaaactatccattcttgtactgtcaagtttgtgggatccccttccaaatgtgtctgattcagctcatactactgatgagtttaacttttttgaaggcacaatcccatcagagatcaccaggcttcctagtttgagaatgatatgggctcccaggtcaactctttcaggaaaatttcctggcagttggggtgcttgtgacaatttggagatcgtgaacttggctcaaaattattatactggagtgattcctgaggaattgggtagctgccagaagttgcattttcttgacttgagctcaaataggctgactggacagcttgttgagaaactgccagtcccttgcatgtttgtgttcgatgtgagtgggaattatctctctggttcaattcccaggttttccaattacagttgtgctcatgttgtttccagcggtggagagccatttgggccctatgatacatcatctgcatatctagcacatttcaccagtagaagtgttctagacactacattatttgcaggtgatggtaaccatgcagtatttcataatttcggtgttaacaacttcacgggaaatttaccgccttccatgctaattgcacctgaaatgttaggcaaacaaattgtatacgcatttcttgctggtagtaacaggtttactggaccttttgctggtaacttgttcgagaaatgtcatgaattgaatggaatgattgttaatgtaagcaataatgcgttgtcaggtcaaatcccagaggatattggtgcaatttgtgggtctcttaggctgttggatggatccaaaaatcagattgttgggacagtccctccgagtttagggagtctggtttcattagttgctctcaatttaagttggaaccacctgcgaggtcagattcctagcagacttggccagataaaggatctcagttacctctctttggctggcaataatctggttggcccaatcccctcaagttttggccaattgcactctttagaaacgcttgaactttcttcgaattctttgtctggtgaaattccaaataatctggtaaatttgaggaatttgacttcccttcttctgaacaacaacaatttatcagggaaaataccttcaggcttggccaatgtgaccacactggcagcatttaacgtttctttcaataatctgtctgggccactgcctcttaacaaagatttgatgaagtgtaatagtgttcagggaaacccctttctgcaatcgtgccatgtattttctctatcaacaccttctacagatcagcagggaagaataggggactcacaagattctgctgcgtctccttcaggttcaacccagaaaggagggagcagcggtttcaactccatagagattgcatccataacatctgcggcagctattgtgtcagttcttcttgctctgatagtcctgttcttttacaccagaaaatggaatccaagatctagagttgctggatctaccaggaaagaagtcacagtgtttacagaagttccggttcctttaacatttgaaaatgtagtgcgggccacagggagcttcaatgcaagcaattgcataggcagtggaggttttggagcaacatacaaagcggagattgcaccagggttcctagtggcagtaaagcgacttgctgtaggacgttttcaggggattcaacagtttgatgcagaaatcagaactctggggaggcttcgacatccaaacctcgtaactctgataggatatcataatagtgaaacagaaatgtttctgatctataactatttgccaggtggtaatttggaaaagtttattcaggagaggtctacaagggctgtggactggagggttcttcacaagattgctttggatgtagcccgtgcacttgcttacctgcatgatcagtgtgtaccacgtgtgcttcatcgtgatgtgaagccgagcaacattttattggatgaggagtataatgcatatttatctgattttggtttggctagattactgggaacttcagagacccatgcaactactggtgtggcgggaacttttggatatgttgctcctgaatatgccatgacttgccgcgtctcggacaaggctgatgtctacagttatggggttgtgttgcttgagttaatatcagataagaaagcactagatccgtctttctcttcttatggaaatggattcaatattgtagcttgggcatgcatgcttttacgcagggccgtgctaaggagttctttacggctggtctatgggattcaggtccacatgatgatttggatgaggtcctacacttggcagtggtctgcacggttgactctctttctactagaccaacaatgaagcaagtagtaagacggttgaagcaacttcaacccccgtcgtgttagctgcggcatgtgttttggataggatatggtttagcccaattgtaatnttaaaacttgcccttgatagtaaggtgtatttgggtgtctcgtattaggttcagatttgtatttgtagcctgcttgtgaattgtagtatatagccagcccccnatttttccnatgtcatgtcccntaattagggggtgtgcagattcttctngcagaagagtgcagatacttgtcttcaacatgtaccnacatttttttttgtttgttaaataagagcaaaaaataggaaccaaaaaaaaaaaaaaaaaaaaaaaaaaaaaaaannngc

SEQ ID NO: 20 - protein, 1161 aa
DSLCLSLFAAPKSRVRARVFEFQNPISGSYNLHIQGEFVSVAFFEDPFGVLLMGRCCFVIKWYYHDIPLKVFLILCVFFLVHGYALSSDSDKSALLELKASFSDSSGVISSWSSRNNDHCSWFGVSCDSDSRVVALNITGGNLGSLSCAKIAQFPLYGFGITRVCANNSVKLVGKVPLAISKLTELRVLSLPFNELRGDIPLGIWDMDKLEVLDLQGNLITGSLPLEFKGLRKLRVLNLGFNQIVGAIPNSLSNCLALQIFNLAGNRVNGTIPAFIGGFEDLRGIYLSFNELSGSIPGEIGRSCEKLQSLEMAGNILGGVIPKSLGNCTRLQSLVLYSNLLEEAIPAEFGQLTELEILDLSRNSLSGRLPSELGNCSKLSILVLSSLWDPLPNVSDSAHTTDEFNFFEGTIPSEITRLPSLRMIWAPRSTLSGKFPGSWGACDNLEIVNLAQNYYTGVIPEELGSCQKLHFLDLSSNRLTGQLVEKLPVPCMFVFDVSGNYLSGSIPRFSNYSCAHVVSSGGEPFGPYDTSSAYLAHFTSRSVLDTTLFAGDGNHAVFHNFGVNNFTGNLPPSMLIAPEMLGKQIVYAFLAGSNRFTGPFAGNLFEKCHELNGMIVNVSNNALSGQIPEDIGAICGSLRLLDGSKNQIVGTVPPSLGSLVSLVALNLSWNHLRGQIPSRLGQIKDLSYLSLAGNNLVGPIPSSFGQLHSLETLELSSNSLSGEIPNNLVNLRNLTSLLLNNNNLSGKIPSGLANVTTLAAFNVSFNNLSGPLPLNKDLMKCNSVQGNPFLQSCHVFSLSTPSTDQQGRIGDSQDSAASPSGSTQKGGSSGFNSIEIASITSAAAIVSVLLALIVLFFYTRKWNPRSRVAGSTRKEVTVFTEVPVPLTFENVVRATGSFNASNCIGSGGFGATYKAEIAPGFLVAVKRLAVGRFQGIQQFDAEIRTLGRLRHPNLVTLIGYHNSETEMFLIYNYLPGGNLEKFIQERSTRAVDWRVLHKIALDVARALAYLHDQCVPRVLHRDVKPSNILLDEEYNAYLSDFGLARLLGTSETHATTGVAGTFGYVAPEYAMTCRVSDKADVYSYGVVLLELISDKKALDPSFSSYGNGFNIVAWACMLLRRAVLRSSLRLVYGIQVHMMIWMRSYTWQWSARLTLFLLDQQ

SEQ ID NO: 21 - DNA (genomic), 473 bp; single-stranded, linear
  - 1..473: /note= “TRK3 Xa21 gene from tomato”
tcaagcaacaatttgtcaggccagattcccaagtccttgaggaatcttgaacatctcatgtatttcaatgtctcgttcaatgggctcatgggtgaaattccagatggagggccattcgtaaattttacagctgaatcattcatgggtaaccctgcattatgtggatcatcacgcttccgtgtgatgcaatgcagagtcactagtcttgaaagaaaaggaaagagtagagtcttaacttctgttcttgcatcagcttcctcaggagttgtagtcacgaccattttcatcatttggtttctgaaatgccgaaaaaggagtacggaacttcctctagttgatacatttggtcaggtacataagaggatttcgtactatgatattcctcaagggacaaacagctttgatgaagcaaacttgattggaagggggagccttggtttggtctacaaaggaaagcttgaaaatcctaagcg

SEQ ID NO: 22 - protein, 159 aa
  Protein 1..159: /note= “TRK3 Xa21 gene from tomato amino acid sequence”
SSNNLSGQIPKSLRNLEHLMYFNVSFNGLMGEIPDGGPFVNFTAESFMGNPALCGSSRFRVMQCRVTSLERKGKSRVLTSVLASASSGVVVTTIFIIWFLKCRKRSTELPLVDTFGQVHKRISYYDIPQGTNSFDEANLIGRGSLLGLVYKGKLENPKR

SEQ ID NO: 23 - DNA (genomic), 1438 bp; single-stranded, linear
  CDS 2..1438: /note= “TRK4 Xa21 gene from tomato”
agcttggatcaatttaacaatgataaactgcaatttagctggtcctttgcctgaatttcttggaactatgtcttctttagaggttttgttgttgtctacaaataggctttcagggcctattccaggtactttcaaggatgcagtgctgaagatgctttggttgaacgatcagtcgggtgatggtatgagtggttcaatagatgttgttgcaactatggtatcacttacacatctttggcttcatgggaatcaattttcaggtaaaatcccagtagagattggtaatctaacaaatctgaaggatctcagtgtgaatacaaataaccttgttggattaatccctgaaagtttagctaatatgccattagacaatcttgatttgaataataatcattttatgggaccagttcctaagttcaaggctactaatgttagttttatgtccaactctttttgtcaaaccaaacaaggagcagtatgtgcccctgaggttatggcacttttagagtttcttgatggggtgaattatccttctaggcttgttgaatcatggtctggaaacaacccttgtgacggacgttggtggggaataagctgtgacgataaccaaaaagttagtgttataaacttgcccaagtctaatctttccgggaccttgagtccttcaatcgcgaaccttgaaaccgttactcacatttatcttgaatcaaataatctttctggttttgttccatctagttggactagtttgaaatctctgtctattcttgatttgagtaataacaatatttccccacctttgcctaaatttaccacccctttgaaacttgttctaaatggaaatccaaagctgacttctaatcctcctggagcaaatccttcaccaaacaacagcacaactcctgcagattcacccacgtcgtctgtaccatcttcacgacccaacagttcaagctctgtgatctttaaacccagtgaacagtcacccgagaaaaaggactcaaagtcaaagatagctatagttgtggttcctattgctggttttctacttttggtttgtcttgctattccactgtacatttatgtctgtaagaagagtaaagataagcatcaagctccaactgctcttgtggttcatcctagagatccgtctgattcggataatgtagtcaagattgcgattgccaatcagactaatggaagtctttccacagtaaatgcaagtggttctgctagcatacacagtggtgaatcccatttgatcgaagctgggaatttgctcatatcggttcaagtacttcggaatgtgactaagaatttttctccggaaaatgaacttggacgtggtggttttggtgtggtttataagggagaattagatgatgggacacgaatcgctgtcaaaagaatggaggctggtattgttagcaacaaagct

SEQ ID NO: 24 - protein, 479 aa
AWINLTMINCNLAGPLPEFLGTMSSLEVLLLSTNRLSGPIPGTFKDAVLKMLWLNDQSGDGMSGSIDVVATMVSLTHLWLHGNQFSGKIPVEIGNLTNLKDLSVNTNNLVGLIPESLANMPLDNLDLNNNHFMGPVPKFKATNVSFMSNSFCQTKQGAVCAPEVMALLEFLDGVNYPSRLVESWSGNNPCDGRWWGISCDDNQKVSVINLPKSNLSGTLSPSIANLETVTHIYLESNNLSGFVPSSWTSLKSLSILDLSNNNISPPLPKFTTPLKLVLNGNPKLTSNPPGANPSPNNSTTPADSPTSSVPSSRPNSSSSVIFKPSEQSPEKKDSKSKIAIVVVPIAGFLLLVCLAIPLYIYVCKKSKDKHQAPTALVVHPRDPSDSDNVVKIAIANQTNGSLSTVNASGSASIHSGESHLIEAGNLLISVQVLRNVTKNFSPENELGRGGFGVVYKGELDDGTRIAVKRMEAGIVSNKA

SEQ ID NO: 25 - DNA (genomic), 686 bp; single-stranded, linear
  - 1..686: /note= “TRK5 3′ from tomato”
tgagtcagcatttgtgttgaatggcaagaattaggctaacaaacctcttaacttggaaggcaaagggtaacgatagcattggatgtggcaagaggggttgaatacctttcatagcttggcacaacaaagtttcattcatagagatttaaaaccgtcaaacatccttctcgggagacgacatgagagccaaggttgcagattttggtttggttaagaaatgcccctgatggaaagtattctgtggagacacgtttggctggaacttttggctatcttgcacctgaatacgcgggtaagcatttccactccttgtatcattacatcttttttaaccgagaactgatttgtatgcccctagctgtaaactttagatcttagtaaattttaaaattattcgatcatctgcattgctaggcattctggtcttcattacatttagagacatttggcacatagtgttaatactgaggcaatctccatgcgtaaatgctgctttgtcatttttatcgaactcatgaactatatgctactattataaaagataagtttccatttcgtgtggatcagcatctgaattattatcctattacatctcctctttgttacagctactggacgagtcacaaccaaagtagacgtttacctttggcgttgttttgatggagatcattactggtagaaaagct

SEQ ID NO: 26 - DNA (genomic), 407 bp; single-stranded, linear
  - 1..407: /note= “TRK5 5′ from tomato”
ctttctccaactccttctggttggtaagttctaaggctttctgttcttggactaatgtatttgtgacaagtcttcatctacagtaacttcaattaatcttgattctcaatctgtttctggttctttaccttctganattagtcaactttctaatcttaaaaccctttcacttcaaaaaaacaaactttctggccctttaccttcttttgccaacatgtcaaaattagctgatcttttcttggacaataaccaattcacttctgttcctcaagatttccttttgggggttcctagtttagtaactttaagcattantgaaaatgcgggactctctccttggcaaatacctatgtatttaactganagtaccaaatttgggatctctatatgctagtaatgcaagtatt

SEQ ID NO: 27 - protein, 131 aa
  Protein 1..131: /note= “TRK5 5′ amino acid sequence”
LSPTPSGWSTSKPFCSWTNVICDKSSSTVTSINLDSQSVSGSLPSDISQLSNLKTLSLQKNKLSGPLPSFANMSKLADLFLDNNQFTSVPQDFLLGVPSLVTLSISENAGLSPWQIPMYLTESTKFGISIC

SEQ ID NO: 28 - DNA (genomic), 865 bp; single-stranded, linear
  - 1..865: /note= “TRK6 3′ from tomato”
gcttatgacagtcattgatccaactctagacgtgaaagatgagataaccgagagcatttccaccttagctgaacttgctggtcattgcactgcaagagaacctggtcaacggccagatatgggccacgctgtaaacgtgctgtccccacttgttgagaaatggaagcctcttgaggatgatccagcaggactattgtggtatcgactacagtcttcccctcaatcaaatggtcaagggttggcaagaatcggaaggaaaagacttaagttacgtggatctcgaggacagtaagggcagtatcccagcaagaccaactggatttgcagattcatttacatcagctgatggtagataatggaggtacttctatgtagtagatgtagatatcaattttctttgtattgtattgagattttgatcgtattttccacgtgccttcgctcatttctcccccttcaatgtgaatgtattagaaatttaaactatgtgtagcctcagttccttctgtagatataaaatagcggtgaaggagaactatacatcgatcagttgaactcctcgctcagtcactattttcatcttctactatggtgagatttaagagcatttttttcacctttgcctattttcgtcgtttagttgtgctgcttctgaagctctgtgctgaatacatactgagcggtgaagtagcccggtatatagaatatgtcgtttgattcgaagaatctaaccatatagcttcttccttctgctcaaatagggcaatacttggagaagataacccttatgtgtcctcatcatttgttgttttgagaaaaaagtatgatatatgttgccttagactcttcgaaaaggtattgtcat

SEQ ID NO: 29 - DNA (genomic), 769 bp; single-stranded, linear
  - 1..769: /note= “TRK7 3′ from tomato”
aagctttcgattctccctatatgtctctcatgttaactaatatagcttgataaacctagtaaattcttcatactttgtgtatatagtgatatcgaaacgtgagatgcagcctttatccgccttgttcttttttcttggcactctacttgttctgttacttctcatatttatcttatgcaagtttttcaaaccaggaaaactgacagctttgattactagcacatttaagcctcttccaggttttgttttgtttcgaattcatgttactttgagtatcttattcgttattttaaaatatgtcttttttcggatgatacagagatgagtaatgctgttagtgtacatcttcatacaataagctactttaactttcatacattggagaaagctacgaagaattttcactcggataacctccttggatgtggtggatttggtccggttttcctggtaaggtcaatgtgtcttaacgagttgtaaaatccgtttttataattagatgcagaggttaagaattggccctagtgttttttcttaggggaagttaggagatggacaattagttgctgtcaagaagttgtctgttgacaaatcgcagcaaggggaccgggagtttcttgctgaggtacggatgataacaagtatacaacacaagaaccttgtccgccttcttggttgtttgttcaaaagggggatcagaggccttctcgtgtaacgaataacatgaagaataaggaggcttgggaccaaatactatattggtatttat

SEQ ID NO: 30 - DNA, 29 bp; single-stranded, linear
  - 1..29: /note= “L3a LRR region primer”
tcaagcaacaatttgtcaggncarathcc

SEQ ID NO: 31 - protein, 4 aa
GQIP

SEQ ID NO: 32 - DNA, 33 bp; single-stranded, linear
  - 1..33: /note= “K2a kinase region primer”
taacagcacattgcttgatttnanrtcncgrtg

SEQ ID NO: 33 - protein, 5 aa
HCDIK

SEQ ID NO: 34 - DNA (genomic), 40 bp; single-stranded, linear
cggtgcccatcgtccaccatgacagcttgaatcttataac

SEQ ID NO: 35 - DNA (genomic), 40 bp; single-stranded, linear
cggtgcccatcgtccaccacgacggcggcggcgaacgccg

SEQ ID NO: 36 - DNA (genomic), 40 bp; single-stranded, linear
cagctgccattttccacaaagacagcttgaatcttataac

SEQ ID NO: 37 - DNA, 33 bp; single-stranded, linear
  - 1..33: /note= “K1a kinase region primer”
cgccttaggattttcaagctttccyttrtanac

SEQ ID NO: 38 - DNA (genomic), 33 bp; single-stranded, linear
  - 1..33: /note= “K2b kinase region primer”
taacagcacattgcttgatttnanrtcrcartg

SEQ ID NO: 39 - DNA, 33 bp; single-stranded, linear
  - 1..33: /note= “K2c kinase region primer”
taacagcacattgcttgatttnanrtcyctrtg

SEQ ID NO: 40 - DNA, 18 bp; single-stranded, linear
  - 1..18: /note= “L3u primer”
tcaagcaacaatttgtca

SEQ ID NO: 41 - DNA, 21 bp; single-stranded, linear
  - 1..21: /note= “K1u primer”
cgccttaggattttcaagctt

SEQ ID NO: 42 - DNA, 18 bp; single-stranded, linear
  - 1..18: /note= “K2u primer”
taacagcacattgcttga

SEQ ID NO: 43 - protein, 41 aa
SNCGSTYCMLGNTPFVSFAKVKSQINLSEVVAKLICNYVRS

SEQ ID NO: 44 - protein, 29 aa
SGRSSKYLLHLLLTFDIFQSICNFKSLVI

SEQ ID NO: 45 - protein, 17 aa
GMISNYILSTWNSVLDV

SEQ ID NO: 46 - protein, 42 aa
  Modified-site 14: /product= “OTHER” /note= “Xaa = His or Arg”
  Modified-site 15: /product= “OTHER” /note= “Xaa = Ile, Arg or Met”
  Modified-site 16: /product= “OTHER” /note= “Xaa = Gln, Pro, Lys or Thr”
  Modified-site 20: /product= “OTHER” /note= “Xaa = Pro or Leu”
  Modified-site 21: /product= “OTHER” /note= “Xaa = Phe, Leu or Ile”
  Modified-site 22: /product= “OTHER” /note= “Xaa = Gln or Leu”
  Modified-site 23: /product= “OTHER” /note= “Xaa = Lys or Thr”
  Modified-site 24: /product= “OTHER” /note= “Xaa = Ser, Thr, Ala, Asp or Glu”
  Modified-site 25: /product= “OTHER” /note= “Xaa = Ser or Thr”
  Modified-site 27: /product= “OTHER” /note= “Xaa = Pro, Arg, Ser or Thr”
  Modified-site 29: /product= “OTHER” /note= “Xaa = Phe, Ser, Leu, Ile, Thr or Met”
YPRDVWRISKESRXXXLIPXXXXXXRXRXPPILTDSRSRRHQ

SEQ ID NO: 47 - protein, 15 aa
SNFNPRRVSCGMCFG

SEQ ID NO: 48 - protein, 9 aa
  Modified-site 5: /product= “OTHER” /note= “Xaa = Phe, Leu, Ile or Val”
PNCNXKTCP

SEQ ID NO: 49 - protein, 14 aa
GVFGCLVLGSDLYL

SEQ ID NO: 50 - protein, 5 aa
PACEL

SEQ ID NO: 51 - protein, 13 aa
  Modified-site 6: /product= “OTHER” /note= “Xaa = Arg, Gln, Pro or Leu”
  Modified-site 9: /product= “OTHER” /note= “Xaa = Arg, Gln, Pro or Leu”
YIASPXFFXCHVP

SEQ ID NO: 52 - protein, 32 aa
  Modified-site 21: /product= “OTHER” /note= “Xaa = Tyr, His, Asn or Asp”
LGGVQILLAEECRYLSSTCTXIFFCLLNKSKK

SEQ ID NO: 53 - protein, 14 aa
  Modified-site 13: /product= “OTHER” /note= “Xaa = Ile, Met, Thr, Asn, Lys, Ser or Arg”
  Modified-site 14: /product= “OTHER” /note= “Xaa = Cys, Arg, Ser or Gly”
EPKKKKKKKKKKXX